(19) United States
(12) Patent Application Publication
(10) Pub. No.: US 2026/0309662 A1
(43) Pub. Date: Oct. 8, 2026

(54) BREASTMILK SENSING DEVICE AND SYSTEM FOR MONITORING BREASTFEEDING EFFICIENCY

(71) Applicant: MYMILK LABORATORIES LTD., HERZLIYA (IL)

(72) Inventors: Sharon Rachel Haramati, Glil Yam (IL); Ravid Shechter Ushpizin, Glil Yam (IL)

(21) Appl. No.: 19/483,379

(22) PCT Filed: Jun. 6, 2024
(86) PCT No.: PCT/IL2024/050566

§ 371 Date: Nov. 11, 2025

(30) Foreign Application Priority Data
Jun. 6, 2023 (IL) 303514

(51) Int. Cl. G01N 27/06; A61B 10/00; G01N 33/04

(52) U.S. Cl. CPC G01N 27/06; G01N 33/04; A61B 10/0045

(57) ABSTRACT

The present invention pertains to a system for monitoring breastfeeding in a human subject, the system comprises a monitoring compartment, and a milk sample holding chamber. The monitoring compartment comprises a sensing module with a set of two electrodes for measuring conductivity, a volume sufficiency sensor, and a temperature sensor. The monitoring compartment comprising a processing unit in data communication with the sensing module, the processing module configured for processing the conductivity, temperature and volume sufficiency of the milk sample and for providing a milk status value relating to the breastfeeding efficiency of the subject. The processing unit comprises a shell housing comprising a shell body and an open shell base, wherein the sensing module is positioned within the shell housing such that at least the sensing portion thereof extends down vertically from the body of the shell housing towards the open shell base. The sample holding chamber comprising at least one sample cavity sized and shaped to hold a predetermined milk sample volume, wherein the monitoring compartment couples with the at least one sample holding chamber so that the open base of the shell surrounds the sample holding chamber to create a milk sample enclosure such that at least part of the sensing portion of the sensing module is positioned within the sample cavity. The system is configured to monitor milk at all stages of breastfeeding and provide at least one status value relating to the breastfeeding efficiency in the human subject.

### FIELD OF THE INVENTION

The present invention pertains to a system, a device, and a method for evaluating, and monitoring of breastfeeding status and progress in a lactating subject, utilizing a milk sample sensing system.

### BACKGROUND OF THE INVENTION

Breastmilk is the gold standard in infant nutrition, offering multiple positive effects on the mother's and baby's health and wellbeing. Although most mothers initiate breastfeeding, over 60% of mothers report they stop breastfeeding earlier than intended, primarily due to issues with milk supply. In recent years, mobile applications and/or sensors for tracking breastfeeding sessions, identifying which breast was used, and estimating milk amounts per feed have been gradually adopted by users. Additionally, questionnaire engines and platforms for connecting a lactating person to a healthcare provider, such as a lactation consultant (in-person or remotely) have also become popular.

Milk supply establishment is a highly regulated bodily process involving synchronization of hormonal, physiological and behavioral elements. Due to the complexity of this process, the underlying causes of milk supply issues are often under-evaluated, causing delays in behavioral or medical treatment attempts. This can result in early cessation of breastfeeding, and/or infant failure to thrive.

Secondary lactation insufficiency, often caused by ineffective or infrequent milk removal, many times impedes optimal infant growth and breastfeeding exclusivity. Secretory activation, also known as the onset of copious milk secretion or lactogenesis II, is a critical phase in the lactation process where the mammary glands begin to produce a significant volume of milk. Inadequate progress in secretory activation can lead to insufficient milk production, infant weight loss, and premature breastfeeding cessation. Early intervention during lactogenesis, the complex mammary gland development and milk secretion process, can enhance milk supply.

For over 70 years, researchers have sought for biomarkers in human milk that can indicate lactation progress. Human milk electrolytes including sodium dynamics and conductivity have been identified as biomarkers for indicating adequate or inadequate secretory activation and predictors of early breastfeeding termination. Research had suggested that improved biomarker dynamics and milk supply can be facilitated by early and frequent breast stimulation and efficient latch and breast emptiness.

Despite their significance, practical large-scale use of breastfeeding biomarkers is hampered by lab-based testing methods. Further, albeit recent technological advancements, and personnel support, there remains a crucial need for a device that can readily monitor breastfeeding and provide real-time and early-stage feedback. Early lactation support, aided by telehealth and mobile health incorporating at-home device technologies, can drastically influence breastfeeding success.

PCT/IL2020/050106 to the inventors of the present invention pertains to methods and devices for determining inadequate lactation in female subject.

There is nevertheless an unmet need in providing a system for early and direct estimation of breastfeeding effectiveness in a lactating woman. There is a need in a system that can provide evaluation of the breastfeeding status via milk biomarker sensing to thereby allow early intervention and breastfeeding correction. There is a need for a system that would allow for accurate measurement in very small milk sample volumes as milk is produced in very limited quantities in the first days postpartum, which is the most critical period for setting breastfeeding success. Additionally, the system should be easily operated, robust to possible errors, and be intuitive to operate. The system should also provide values that can be readily and reliably interpreted by non-professional users. The present invention provides and details such a system and methods of use thereof.

### SUMMARY

The current invention pertains to a system, a device and a method for effectively measuring biomarkers in milk. The invention pertains to a system, a device and a system that can afford milk biomarker assessment and monitoring from each breast separately and comparing between them. The herein system, device, and method allows to record and compare between current and previous measurements. The herein system, device, and method further allows a comparison between measured individual milk parameters and relevant population benchmarks of adequately and exclusive breastfeeding mothers to assess milk maturation and progress. A specialized maturation scale (termed herein “milk maturation %, or MM %”) and calculated milk post birth age as early indicators, for breastfeeding effectiveness and progress are afforded. Using the milk maturation % parameter, a subject can be guided and directed toward behavioral and clinical activities for potentially improving breastfeeding experience and success.

Thus, the present invention provides a system that comprises a milk monitoring compartment and a milk sample holding chamber. The milk monitoring compartment comprises a sensing module that includes a set of conductivity electrodes configured to measure milk sample conductivity in tiny milk volumes. The sensing module may further comprise sensing means configured to verify a volume sufficiency of a tested sample in the milk sample holding chamber. The sensing module may further comprise sensing means configured to evaluate the temperature of the tested milk sample and adjust the conductivity thereof to reflect a conductivity at ambient temperature (termed herein a temperature adjusted conductivity). The system further comprises a processing unit and optionally a computing device configured to evaluate and process milk related parameter(s). The processing unit and/or computing device configured to determine a breastfeeding status, i.e., determine if the breastfeeding status is detected as delayed or advanced by processing the temperature adjusted milk sample conductivity and comparing it to a suitably comparable reference population dataset. The system is capable of generating a visual output of the breastfeeding status values, and triggering an alert or a visual output when the measured values are out of a range, exceed, or below a threshold of a corresponding reference dataset. The system is further capable of storing previous measurement(s) of milk related parameters of a female subject and compare between the measurement(s) and thereby detect individual breastfeeding progress rate and identify specific breast-health conditions. The system is further capable of comparing the breastfeeding performance between both breasts. The herein system can be implemented in a mobile remote computing device and/or in a cloud-based computer system.

Advantageously, the herein system was designed to be a robust calibration-free milk sample monitoring system. The system is intended for use with a small human milk specimen. The system may include two identical separate milk sample holding chambers, enabling sampling from each breast separately. A cleaning tool provided with the system allows to clean the milk sensing module that came in touch with milk sample after each milk sample measurement, and the milk sampling chambers can be cleaned independently making the device and milk sampling easily reusable for measuring milk conductivity in both breasts and repeatedly over months.

The invention relates to a system, a device, and a method for evaluating and/or monitoring breastfeeding efficiency in a lactating person. The system includes a milk sample monitoring device comprising one or more processing units and one or more computer readable media storing executable instructions. The instructions, when executed by the processing unit, configure the computer system to perform various functions as set forth.

The instructions, when executed by the processing unit, configure the system to receive a conductivity and/or a resistivity measurement from a sensor configured to make such measurements in a milk sample from at least one breast of a lactating person. The sensor is capable of measuring the conductivity and/or resistivity of the milk sample. Conductivity and/or resistivity are used in the current invention as valid indicators of the maturity of the milk and the lactation status of the mother.

The instructions, when executed by the processing unit, configure the system to identify the indication of breastfeeding status and/or a breast health issue based upon at least one measurement relating to the conductivity level of the sample from the lactating person. The system is capable to identify the conductivity level of the human milk sample and provide an output regarding a particular breastfeeding status value, indicating the maturity of the milk and the lactation status of the mother.

The instructions, when executed by the processing unit, configure the computer system to compare the measured level of a milk related parameter (e.g., conductivity) to a reference population set, generating an alert alarm level when the measured milk related parameter is out of the values of the reference population set. In one or more embodiments, the system compares the conductivity of the human milk sample to a reference dataset which corresponds to the same day or day range. If the conductivity level is outside of the reference population set, the system generates an alert, indicating that there may be an issue with the lactation status of the mother. Various levels of alerts are applicable. In one embodiment, the system includes two or more levels of alerts. For example, the system includes three levels of alerts, with the specific alert chosen by the algorithm based on its relation to the reference set values. In one embodiment, when a maturation parameter is calculated, a first alert is set as any value that is below the 15^{th} percentile for the specific day or day range after birth in the reference population dataset. In one embodiment, a second alert is set to be between the 15^{th} percentile and the 50^{th} percentile as calculated for the specific day or day range after birth in the corresponding population dataset. In one embodiment, a third alert is set to be between the 50^{th} and the 85^{th} percentile as calculated for the specific day or day range after birth in the corresponding population dataset. Values of 85^{th} percentile or above may trigger a fourth type of alert. In addition, the system can determine a milk after-birth age, by identifying the specific day or range of days after birth that corresponds to the measured parameter. This is achieved by matching the measured milk maturation percentage or milk conductivity to a matching/corresponding reference dataset containing similar milk maturation percentages or milk conductivity ranges for each day or range of days after birth. The instructions, when executed by the processing unit, configure the computer system to compare a particular value measured in milk to value(s) from previous sample(s) recorded from the same mother. In some embodiments, the one or more values include but are not limited to previous milk conductivity value(s) from milk sample(s) taken from the same breast, previous milk conductivity value(s) from milk sample(s) taken from the other breast. In one or more embodiment, the herein system, method and device enable to provide a subject an evaluation regarding its particular breast-health condition(s). The system can present a particular status value based on previous measurements of the conductivity of previous samples, including previous milk conductivity values from samples taken from the same breast or the other breast. This allows the system to afford a personalized milk state and thereby indicates a particular lactation status and particular breast-health condition(s) associated with the lactation status of the mother.

The herein system further incorporates means for triggering an output alarm. When the system generates an alarm, or a certain level of alarm/alert, it also triggers an output data, alerting the mother or a healthcare personnel about a potential issue with the lactation status of the mother.

The herein system also includes a monitoring compartment that includes a set of two or more conductivity electrodes and one or more temperature sensor(s). In one or more embodiments, the monitoring compartment includes a temperature sensor and a set of two conductivity electrodes. In one or more embodiments, the temperature sensor(s) is placed between the set of two electrodes such that the temperature sensor and two electrodes are in close proximity but spaced apart in all dimensions to avoid direct contact or electrical interference therebetween, such configuration affords accurate and reliable temperature and conductivity measurements in a tiny volume of milk sample within a milk sample holding chamber. The temperature sensor(s) allow(s) adjusting the conductivity measurement to the sample temperature. The monitoring compartment is enclosed within a housing comprising a shell sized and shaped such that it matches a milk sample holding chamber in a manner that enables a specific sample enclosure of a specific pre-determined shape and volume at measurement. The temperature sensor is covered with a heat and electricity conductive casing, or cover made of a conductive material that is electrically wired to one of the conductivity electrodes to facilitate current flow, enabling the closure of an electrical circuit for generating additional output regarding the sufficiency of milk sample quantity for a reliable measurement.

The computer system and device of this invention affords for the first time means for early stage, on site and real time estimation of breastfeeding effectiveness in a lactating woman, utilizing measurements of one or more specific indicators in human milk. This advantageously affords an early detection, and consequently early medical intervention and correction of breastfeeding conditions, specifically in the context of lacking or abnormal breastfeeding establishment. The herein invention assists lactating mothers in achieving a better breastfeeding experience and success by providing means that can monitor and assess the progress of their breastfeeding status. Among the breastfeeding status included herein are secondary lactation insufficiency, low milk supply, delayed secretory activation, delayed lactogenesis, lactation failure, and breastfeeding insufficiency.

In one aspect, the present invention provide a system for monitoring breast milk at various stages of breastfeeding from colostrum through transition to fully mature milk in a human subject, the system comprising: a monitoring compartment for measuring milk related parameters in a sample of milk from the subject, whereas the sample is held in a milk sample holding chamber, the monitoring compartment comprising: a sensing module comprising a set of at least two electrodes for measuring conductivity, a volume sufficiency sensor, and a temperature sensor, the sensing module configured to provide milk related parameter measurement(s) relating to the conductivity, the temperature, and the volume sufficiency of the milk sample, a processing unit in data communication with the sensing module, the processing module configured for processing the conductivity, temperature and volume sufficiency of the milk sample and for providing a milk status value relating to the breastfeeding efficiency of the subject; and a shell housing comprising a shell body and an open shell base, the shell housing encapsulating the sensing module and the processing unit within the shell housing cavity, and wherein the sensing module is positioned within the shell housing such that at least the sensing portion thereof extends down vertically from the body of the shell housing towards the open shell base; and at least one sample holding chamber, the sample holding chamber comprising at least one sample cavity, the sample cavity is sized and shaped to hold a predetermined milk sample volume, and an overflow channel coupled to at least one outlet of the at least one sample cavity for diverting excess sample out of the sample cavity and into the overflow channel; wherein the monitoring compartment couples with the at least one sample holding chamber so that the open base of the shell surrounds at least the interior of the sample holding chamber, forming a milk sample enclosure such that at least part of the sensing portion of the sensing module is positioned within the sample cavity, and wherein the system is configured to monitor milk at all stages of breastfeeding and provide at least one status value relating to the breastfeeding efficiency in the human subject.

In one or more embodiments, the milk sample enclosure is configured to hold a maximal volume of milk sample of about 500 microliters. In one or more embodiments, the milk sample enclosure is configured to hold between about 200 microliters and up to about 500 microliters of milk. In one or more embodiments, the enclosure formed is configured to hold a milk volume of up to about 450 microliters, up to about 400 microliters, up to about 350 microliters, or up to about 250 microliters. Each possibility represents a separate embodiment of the invention.

In one or more embodiments, the enclosure is defined by the walls of the milk sample cavity and a top roof of the shell base, wherein the enclosure, when formed, accommodates a volume of milk in which the entire sensing portion of the sensing module is immersed within the sample cavity.

In one or more embodiments, the at least one sample holding chamber comprises a base, the base comprising a walled ridge to receive the perimeter of the open base of the shell of the monitoring compartment and couples the monitoring compartment to the at least one sample holding compartment.

In one or more embodiments, the conductivity measurement of the milk sample is adjusted according to the milk measured temperature, affording a conductivity output that corresponds to a fixed predetermined temperature, such as ambient temperature.

In one or more embodiments, there are two electrodes, the two electrodes are disposed adjacent to each other, and the temperature sensor is disposed between the two electrodes to thereby form a triangle like structure in a compact configuration for optimal coupling and fit within the milk sample cavity.

In one or more embodiments, the system comprises at least one temperature sensor and at least one set of two conductivity electrodes, wherein the temperature sensor is shorter in length or extends less into the cavity of the sample chamber than the conductivity electrodes.

In one or more embodiments, the volume sufficiency sensor comprises a conductive casing covering the temperature sensor, the casing is electrically wired to one of the conductivity electrodes, wherein the conductivity electrodes are immersed deeper into the milk sample than the temperature sensor and casing, and wherein when the conductive casing is in contact with the milk sample, the circuit is closed between the conductive casing and the at least one conductivity electrode, facilitating a sufficient sample volume signal and when the conductive casing does not contact a volume of milk sample, the circuit between the conductive casing and the conductivity electrode is not closed facilitating an insufficient sample volume signal.

In one or more embodiments, the sensing module extends downwardly from a protruding bulge positioned at the center of a roof of the open shell base, whereas when coupled to the milk sensing chamber the bulge fits to close the opening of the milk chamber and pushes excess sample through the outlets to the channel.

In one or more embodiments, the processing unit is configured to execute computer readable instructions to cause the monitoring device to measure the milk related parameters, analyze the measured parameters, trigger a visual and/or an audio output relating to the measurements, and provide insights for improving breastfeeding efficiency, within up to a few minutes.

In one or more embodiments, the monitoring compartment comprises a communication module configured to transmit the at least one milk related parameter and status values to an external computing device configured to receive the measurements from the monitoring compartment, to display, store, analyze the data and provide insights for improving breastfeeding efficiency.

In one or more embodiments, the computing device is in communication with the system and wherein the computing device is at least one of a smartphone, a laptop, a computer, a tablet and a smartwatch, the computing device comprising a designated application program for monitoring and displaying milk related parameters and data communicated from the monitoring device.

In one or more embodiments, the computing device is configured to provide for one of the breasts, for each breast, or for both breasts in combination, an output value relating to the breastfeeding efficiency status, the output value is selected from: a milk conductivity measurement, a milk maturation score reflecting milk sample maturation percent within the full range from initial colostrum to fully mature milk, a predicted milk sample age after birth, and a breastfeeding progress rate.

In one or more embodiments, the monitoring compartment is configured to provide for one of the breasts, for each breast, or for both breasts in combination, an output value relating to the breastfeeding efficiency status, the output value is selected from: a milk conductivity measurement, a milk maturation score reflecting milk sample maturation percent within the full range from initial colostrum to fully mature milk, a predicted milk sample age after birth, and a breastfeeding progress rate.

In one or more embodiments, the system is configured to compare at least one of the breastfeeding efficiency status values to a reference dataset corresponding to each day or day range after birth and trigger an alarm or a visual output when the at least one breastfeeding efficiency status value is outside of a range, exceeds, or is below a predetermined threshold of the corresponding reference dataset, each with the corresponding alarm or visual output.

In one or more embodiments, values that fail to meet a certain predetermined threshold are indicative of a delayed lactation and wherein values that correspond to a predetermined threshold or are improved values are indicative of an advanced lactation state. In one embodiment, the status value is milk conductivity, wherein a measured conductivity that is above the threshold value for conductivity is indicative of a delayed lactation and wherein a measured conductivity that is below the threshold value for conductivity is indicative of an advanced lactation. In one embodiment, the status value is MM %, wherein a measured MM % that is above the threshold value for MM % is indicative of an advanced lactation and wherein a MM % that is below the threshold value for MM % is indicative of a delayed lactation. In one embodiment, the status value is breastfeeding progress rate, wherein a measured progress rate that is above the threshold value for progress rate is indicative of an advanced lactation progress and wherein a measured progress rate that is below the threshold value for progress rate is indicative of a slower lactation.

In one or more embodiments, delayed lactation comprises a condition selected from low milk supply, delayed lactogenesis, delayed secretory activation, secondary lactation insufficiency, low milk supply, lactation failure, primary lactation failure.

In one or more embodiments, the system is configured to calculate a breastfeeding progress rate by computing the difference and/or ratio between the at least one of the breastfeeding efficiency status values to a previously stored data from the same lactating subject, over a period of a plurality of days to calculate a progress rate, and trigger an alarm or a visual output when the calculated difference and/or ratio is outside of a range or exceeds, or is below a predetermined threshold relative to the reference dataset corresponding to the specific day or day range after birth.

In one or more embodiments, the system is configured to compare the at least one of the breastfeeding efficiency status values between one breast and the other breast and trigger an alert or a visual output when the difference in measurements between the breasts exceeds a predetermined threshold.

In one or more embodiments, the system is configured to calculate the predicted milk sample age after birth by matching the calculated milk maturation score or the measured milk conductivity to a corresponding reference dataset, present the predicted milk sample age to the user and/or trigger an alarm or a visual output when the predicted milk sample age value does not match the actual (real) age of the baby, indicating a delay in breastfeeding progress.

In one or more embodiments, the system is configured to provide an indication of breast health issue. In one or more embodiments, the breast health issue is determined when the difference in status vales between the breasts exceeds a predetermined threshold and optionally also when pain is recorded in the system for the breast that exhibited lower status values relative to the other breast, and wherein the system triggers an alarm or a visual output when there is an indication regarding a breast health issue in the subject.

In one or more embodiments, the breast health issue is selected from a group consisting of breast inflammation, restricted milk flow, breast engorgement, ductal infection, mastitis, breast infection, and breast candida.

In one or more embodiments, the system is further configured to calculate an estimated protein content in the milk sample by an equation computed from a corresponding reference dataset of empirically measured conductivity and milk protein content.

In one or more embodiments, the system is further configured to trigger at least one of an alarm or a visual output indicating a technical error selected from the group consisting of: low battery, a milk sample volume insufficiency, a poor communication signal, a measurement that is outside of a range or exceeds a predetermined threshold values of a corresponding reference dataset, and a difference between a measurement and at least one previous measurements from the same subject that exceeds a predetermined threshold values.

In one or more embodiments, the system is configured to provide insights for improving breastfeeding efficiency according to the measured breastfeeding efficiency status value.

In another aspect, the present invention provides a method for monitoring breast milk sample of a human subject at various stages of breastfeeding from colostrum through transition to fully mature milk, the method comprising: obtaining a small volume of sample of breast milk from a lactating human subject; placing the sample in a cavity of a milk sample holding chamber; measuring in the milk sample a plurality of milk related parameters comprising conductivity and/or resistivity, temperature and sufficiency of volume in the cavity of the milk sample holding chamber; receiving a conductivity and/or a resistivity measurement from a sensor arranged to measure milk related parameters in said milk sample; receiving a milk sample quantity sufficiency signal from a sensor arranged to measure the milk sample level sufficiency within a milk sample holding chamber during a measurement; receiving a milk sample temperature measurement from a sensor arranged to measure the milk sample temperature during a measurement; and identifying at least one breastfeeding efficiency status values based upon a sufficient milk sample level and the at least one measurement relating to the conductivity and/or resistivity of the milk sample, wherein the conductivity and/or resistivity measurement is adjusted to afford a conductivity output that corresponds to a predefined temperature.

In one or more embodiments, identifying a breastfeeding efficiency status value comprises analyzing data related to the milk related parameters and providing an output of the breastfeeding status value.

In one or more embodiments, the breastfeeding efficiency status is selected from a group consisting of: a milk conductivity measurement, a milk maturation score, a predicted milk sample age after birth, and a breastfeeding progress rate.

In one or more embodiments, the method further comprises comparing the at least one of the breastfeeding status values to a reference dataset corresponding to each day or day range after birth and alerting when the at least one breastfeeding efficiency status value is outside of a range, exceeds, or is below a predetermined threshold of the corresponding reference dataset.

In one or more embodiments, the method further comprises indicating a delayed lactation when said values do not meet the predetermined threshold, and indicating an advanced lactation state when the values correspond to the threshold or are improved values comparing to the threshold.

In one or more embodiments, the method further comprising calculating a breastfeeding progress rate by computing the difference and/or ratio between the at least one of the breastfeeding efficiency status values to a corresponding previously stored data from the same lactating subject, in at least two measurements to calculate a progress rate, and triggering an alarm or a visual output when the difference and/or ratio between the least one breastfeeding efficiency status values is outside of a range or exceeds, or fails to meet a predetermined threshold relative to the corresponding reference dataset.

In one or more embodiments, the method further comprising comparing the at least one of the breastfeeding status values between one breast and the other breast and triggering an alert or a visual output when the difference in measurements between the breasts exceeds a predetermined threshold.

In one or more embodiments, the method further comprising calculating a predicted milk sample age after birth by matching the calculated milk maturation score or the measured milk conductivity to a corresponding reference dataset calculated for each day or range of days after birth, presenting the predicted milk sample age to the subject and/or triggering an alarm or a visual output when the predicted milk sample age value does not match the actual (real) age of the baby, indicating a delay in breastfeeding progress.

In one or more embodiments, the method further comprises providing an indication of a breast health issue wherein the breast health issue is determined when the difference in measurements between the breasts exceeds a predetermined threshold and optionally when the subject experiences pain in the breast that exhibits lower breastfeeding efficiency status values relative to the other breast, and wherein the system triggers an alarm or a visual output when there is an indication regarding a breast health issue in the subject.

In one or more embodiments, the breast health issue is selected from a group consisting of breast inflammation, restricted milk flow, breast engorgement, ductal infection, mastitis, breast infection, breast candida, and a combination thereof.

In one or more embodiments, the method further comprises calculating an estimated protein content in the milk sample based on the measured conductivity and a correlation equation between protein content and conductivity measurements. In one or more embodiments, the correlation equation is based on a reference dataset that comprises empirical data of conductivity and protein contents in milk samples.

In one or more embodiments, the method further comprising transmitting the breastfeeding status values and/or measured milk related parameters to a remote computing device for analyzing the measured parameters, providing status outputs, triggering a visual and/or an audio output relating to the measurements, and providing insights for improving breastfeeding efficiency.

In one or more embodiments, wherein providing insights for improving breastfeeding efficiency comprise insights on changing at least one of a breastfeeding habit.

In one or more embodiments, the method further comprising triggering at least one of an alarm or a visual output indicating a technical error selected from the group consisting of: low battery, a milk sample volume insufficiency, a poor communication signal, a measurement that is outside of a range or exceeds a predetermined threshold values of a corresponding reference dataset, and a difference between a measurement and at least one previous measurement from the same subject that exceeds a predetermined threshold value. In one or more embodiments, the method further comprising cleaning said sensors between measurements

An aspect of the invention provides a computer system measuring one or more milk related parameters in human milk for monitoring breastfeeding efficiency comprising: one or more processing unit, and one or more computer readable media storing executable instructions wherein execution thereof by the processing unit configure the computer system to perform at least the following: receive any of conductivity or resistivity measurement from a sensor arranged to measure a human milk sample taken from at least one breast of a lactating person, and receive a milk sample quantity sufficiency signal from a sensor arranged to measure the human milk sample level sufficiency during a measurement, receive a milk sample temperature measurement from a sensor arranged to measure the human milk sample temperature during a measurement, and identify a status value based upon at least one measurement relating to the conductivity measurement of the sample from the lactating person, adjusted to the temperature measurement and the milk level sufficiency signal, comparing the adjusted status value to a corresponding reference dataset value for each day or day range postpartum (also referred to as baby age), generating an alarm value when out of one of a stored reference dataset defined for that value, comparing the value to stored data of milk measurements from the same lactating person including but not limited to: previous milk conductivity value from sample taken from the same breast, previous milk conductivity value from sample taken from the second breast, and re-calculating status value as set in the executable instructions, attributing any of breast-health conditions, secretory activation progress, breastfeeding efficiency, and triggering a status value alarm or visual output.

In one or more embodiment, the system further comprising a sensing module that includes a set of two conductivity electrodes and one temperature sensor placed between the two electrodes with an offset, allowing for correcting the conductivity measurement to the sample temperature, wherein at least part of said sensing module enclosed within a shell that is configured to match a milk sample holding chamber in a manner that enables a pre-determined sample enclosure of pre-determined shape and volume at measurement.

In one or more embodiments, wherein the temperature sensor casing is made of conductive material enabling closing a second electric circuit for generating additional output regarding milk sample level.

In one or more embodiments, the milk sample holding compartment is designed to enable the holding of a fixed volume of milk by introducing a set of connected intrusions and protrusions generating a tunnel system, that when introducing the electrode module and shell base pushes the liquid volume to be adjusted to the fixed volume required for reliable measurement.

In one or more embodiments, the status value is determined by the processor by calculating the secretory activation level of the lactating person. In one or more embodiments, the status value is determined by the processor by calculating the secretory activation level of the lactating person by calculation of milk maturation percent parameter. In one or more embodiments, the status value is determined by the processor by calculating the secretory activation level of the lactating person by calculation of the day after birth day-range relative to predetermined reference dataset. In one or more embodiments, a status value is determined by the processor by calculating an estimated breastmilk protein content by interpolation from empirically pre-defined correlation from early postpartum data set of conductivity and protein content in human milk.

In one or more embodiments, at least the sensing module is integrated into a breast milk pump part. In one or more embodiments, at least the sensing module is integrated into a milk flow meter. In one or more embodiments, the sensing module measurement is launched through a separate computer that is in communication with the sensing module described. In one or more embodiments, the status value is provided on a separate computer that is in communication with the sensing module.

In one or more embodiments, the computer is a smartphone with a designated App that is communicating with the sensing module by Bluetooth.

In one or more embodiment, the status value is calculated to integrate additional user-specific input data including but not limited to: breast pain, breastfeeding confidence, breastfeeding assessment score, breastfeeding frequency, with stored thresholds for each health indicator, as set in the executable instructions.

In one or more embodiments, the status value is used by the lactating female subject or lactating person's caregiver for directing behavioral changes.

In one or more embodiments, any of status level, alarm, and visual output is visual elements and text to be presented to the lactating female subject or care giver.

The present invention further provides a human milk sensing system for monitoring breastfeeding efficiency, comprising a shell housing with electrode set, and a human milk sample holding chamber fit to hold a milk sample volume to be in direct contact with the electrodes and electrode set comprising a conductivity sensor and temperature sensor with electrode tips extending out of the computing shell housing and into the milk sensing central cavity of the human milk sample holding chamber when placed on top of the human milk sample holding chamber, and a sample sufficiency sensor built into the structure of the conductivity sensor and temperature sensor, enabling verifying milk sample sufficiency for accurate measurement, and a human milk sample holding chamber designed to be encapsulated by the sensing module's shell housing and hold a pre-defined milk sample volume when connected to the sensing module, and a processing unit within the shell housing configured to execute computer readable instructions that when executed cause the sensing device system to perform the following steps: receive from the conductivity sensor at least a milk conductivity level of the sample, and receive from the milk sample level sensor at least a milk sample level sufficiency signal, output an error value when milk level is not sufficient, receive from the temperature sensor at least a temperature of the sample, and correct the conductivity value according to the temperature measured by the temperature sensor, and identify the status value based upon at least one measurement relating to the temp corrected conductivity level of the sample from the lactating person, and comparing the status value to a reference dataset values for each day or day range postpartum (also referred to as baby age), generating an alarm level when out of one of a stored reference dataset defined for that value type, and comparing the value with stored data of milk measurements from the same lactating person including but not limited to: previous milk conductivity value from sample taken from same breast, previous milk conductivity value from sample taken from the second breast and re-calculating status value attributing any of breast-health conditions, secretory activation progress, breastfeeding efficiency, and trigger a status value alarm or visual output.

### BRIEF DESCRIPTION OF THE DRAWINGS

In order to describe the features of the invention, a more particular description of the invention will be rendered by reference to specific embodiments, which are illustrated in the appended drawings. Understanding that these drawings depict only typical embodiments of the invention and are not therefore to be limiting of its scope, the invention will be described and explained with additional specificity and detail through the use of the accompanying drawings in which:

FIG. 1 illustrates an exemplary system for monitoring the breastfeeding efficiency of a lactating person. The figure shows the overall layout of the system, including a milk sample holding chamber, a monitoring compartment and a computing device, in accordance with an aspect of the present invention.

FIG. 2 illustrates the layout and design of an exemplary milk sample monitoring device comprising a monitoring compartment and an exemplary milk sample holding chamber, in accordance with an aspect of the present invention.

FIG. 2A shows an exemplary milk sample holding chamber configured to match and fit the monitoring compartment, enabling the specific sample enclosure of a specific pre-determined shape and volume at measurement, according to an aspect of the present invention.

FIGS. 2B-2C show an exemplary monitoring compartment comprising a set of two conductivity electrodes and a temperature sensor, and their position within a shell structure, according to an aspect of the present invention.

FIG. 2D illustrates an exemplary encapsulated sensing cell created when the exemplary milk monitoring compartment is placed on top of the exemplary milk sample holding chamber, according to an aspect of the present invention.

FIG. 2E depicts an exemplary processing unit comprising an electric circuit board comprising electrodes inducing a first electrical circuit for conductivity measurement and a second electrical circuit of the temperature sensor with a conductive casing for generating additional data output regarding the milk sample level within the holding chamber. The exemplary circuit board includes a transmitter, a battery, a physical communications port, and a LED, in accordance with an aspect of the present invention.

FIGS. 3A-3C depict a smart phone with an exemplary display interface. The figure shows the alarm output and a display of the system to the user, according to an aspect of the present invention.

FIG. 4 depicts a flow chart illustrating steps in a method of the present invention. The flowchart describes exemplary logics applied to milk sample readings, including technical alerts and visual outputs, in accordance with an aspect of the present invention.

FIGS. 5A-5E depict examples of a system described by the current invention and exemplary App. interfaces in a set of different real cases, in accordance with an aspect of the present invention.

FIG. 6 depicts examples of analysis of data gathered with an example of the system described in the current invention in a set of different real cases, demonstrating the ability of the system to differentiate between breastfeeding status between groups of samples according to breastfeeding exclusivity and breastfeeding problems, in accordance with an aspect of the present invention.

FIG. 7 is a line graph depicting an example of a method of determining protein contents in a milk sample. The method utilizes empirical correlation between human milk conductivity as measured by an exemplary system of the present invention with laboratory grade human milk protein content assessment.

FIGS. 8A-8B show an exemplary system for identifying whether the milk sample level in the milk holding compartment is sufficient for a valid reading by the system of the present invention, in accordance with an aspect of the present invention.

FIG. 9 depicts a flow chart illustrating steps in the method of the invention, in accordance with an aspect of the present invention.

FIGS. 10A-10B depict a cleaning tool, according to an aspect of the invention.

FIG. 11A-C are graphs showing the reliable measurements produced by the device described in this invention in a range of sample temperatures and volumes (A) conductivity of specimens placed in the milk cavity of the milk sample chambers gradually elevated from 200 μl to 450 μl as recorded by the device in two levels of standard conductivity solutions. X-system alerted of insufficient specimen volume for performing measurement. (Ci) Series of conductivity measurements by device vs. specimen temperature in a bath-warmed standard sample left to cool to ambient temperature while a series of measurements are performed on the sample (Starting Temp 39° C., End Temp 30° C.; yellow) and a Fridge-cooled standard sample left to warm to ambient temperature while a series of measurements are performed on the sample (Starting Temp 17° C., End Temp 24.7° C.; blue). Two Room Temp standard samples were included for comparison (26.4° C.; 27.6° C., black). (Cii) Conductivity measurements of two levels of standard materials from 10 different measurement dates performed at various ambient temperatures. The X-axis represents the sample temperature as measured by the device.

FIGS. 12A-C shows device measurements are accurate stable and reliable over time. (A) Linear regression of conductivity to KCl molarity in the measuring range of 10-100 mM. (B) Levey-Jennings chart of the quality control data logged by one of the devices tested for stability, with two levels of conductivity standard solution (Level 1 (1413 μS/cm) and Level 2 (5.00 mS/cm)) collected over a 2-month period. The middle line is the mean, and the dashed line measures the distance from the mean to the standard deviation. (C). Test-retest analysis to assess the consistency of the measurement set-over devices by comparing conductivity records in a set of 28 mothers' milk specimens.

FIGS. 13A-B shows validation of the device compared to Na+ ion selective electrode instrument (A) correlation and regression analysis comparing calculated sodium equivalent (Na+ Eqv), based on conductivity measurement by the device converted by calibration equation, against real sodium ion levels (Na+) as recorded by ion selective electrode (LaquaTwin, Horiba), recorded in a dataset of breast milk (N=132). (B) Bland-Altman plot plotting the difference between paired measurements (y-axis) over the average of these measurements (x-axis).

FIGS. 14A-C show plots of Milk maturation percent (MM %) distribution in a dataset of a predominant breastfeeding population. A dataset of 507 scans recorded from exclusive breastfeeding or predominant mother's milk (>80% daily feeds), with no problems at the time of the scan were included. (A) Dot plot chart of MM % to day from birth at scanning. The upper chart shows the full scale up to 300 days (N=507); the lower chart enlarges the 0-60 days snapshot (n=463). (B) Box plot presentation of the data distribution and quantile limits (+1.5 IQR) for each day postbirth (0-60 days). (C) MM % to day postbirth (0-60 days) chart with ranges set by the data set percentile array, where the lower full line depicts the 15th percentile, the middle dashed line depicts the 50th percentile, and the upper dotted-dashed line depicts the 85th percentile.

### DETAILED DESCRIPTION OF THE PREFERRED EMBODIMENTS

It is understood that the invention is not limited to the particular methodology, systems, devices, apparatus, items or products etc., described herein, as these may vary as the skilled artisan will recognize. It is also to be understood that the terminology used herein is used for the purpose of describing particular embodiments only and is not intended to limit the scope of the invention. The following exemplary embodiments may be described in the context of exemplary human milk evaluation systems and devices for ease of description and understanding. However, the invention is not limited to the specifically described products and methods and may be adapted to various applications without departing from the overall scope of the invention.

The device, system and methods of the present invention have many advantages. The system can provide an early indication of breastfeeding issues or conditions. The system can also provide an early indication of a lactation problem of the mother. These indications can lead to early intervention to ensure improved lactation and thriving of the milk recipient. Advantageously, the invention can be used for reliable measurement on a very small volume samples providing a method of monitoring the status and progress of breastfeeding very soon after birth when the available volume of produced milk is typically very limited. The system is designed to be reliable, easy and quick to use in a home environment by a non-professional user, without the need for a specialized setting, manual calibrations, or specialized tools. The system is designed to prevent recording error measurements due to insufficient sample volume or temperature outside of the system's intended use that may affect the reliability of the results, thus providing a seamless level of error proofing for laymen use. The system and results are intuitive and can be used without the need for medical expertise and therefore can be used by an individual, for immediate analysis and results supporting self-management. The system enables routine use by tracking each breast separately, repeatedly and reliably monitoring milk supply status and daily progress. The system also facilitates comparison between breasts and detects small changes thereby providing early indications for breast health issues that may develop into breast inflammation, helping the mother manage these conditions in advance. Additionally, the system supports unique, data driven remote lactation care, for mothers at home, and enables objective repeated measurement provided for health practitioners for improving case management, prioritize care, monitoring intervention effectiveness and to monitor breastfeeding progress.

As used herein the terms ‘a’ and ‘an’ may mean ‘one’ or ‘more than one’.

All ranges disclosed herein include the endpoints. The use of the term “or” shall be construed to mean “and/or” unless the specific context indicates otherwise.

Each of the following terms: ‘includes’, ‘including’, ‘has’, ‘having’, ‘comprises’, and ‘comprising’, and, their linguistic, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof.

The term ‘consisting essentially of” as used herein means limited to the specified elements and those that do not materially affect the basic and novel characteristic(s) of the claimed invention.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

The term ‘about’, in some embodiments, refers to ±30% of the stated numerical value. In further embodiments, the term refers to ±20% of the stated numerical value. In yet further embodiments, the term refers to ±10% of the stated numerical value.

Embodiments of the present invention include systems, methods, and devices that monitor breastfeeding by milk sensing. In particular, an embodiment of the present invention pertains to monitoring mother's breast milk conductivity level and calculated maturation % and provides an output alert when a potential breastfeeding condition or issue is detected. The principles and operation of a system and a device, such as a breastfeeding monitoring system and device for monitoring breastfeeding efficiency, as well as methods of use thereof according to the present invention may be better understood with reference to the figures. The figures show non-limiting aspects of the present invention.

As used herein, the milk sample monitoring device affords measuring one or more milk related parameters. The one or more milk related parameters include milk conductivity measurement. The herein system can measure various ranges of milk conductivities. For example, the system may be configured to measure conductivity in milk at values of 1000-10000 uS/cm, and specifically at values of 1500-5000 uS/cm. The one or more milk related parameters include milk resistivity measurement. The one or more milk related parameters include milk temperature measurement. The herein system can measure various milk temperatures. For example, the system may be configured to measure temperatures of 15 degrees Celsius-40 degrees Celsius. The one or more milk related parameters include sufficiency of milk sample for reliable measurement. By sufficiency of milk sample, it is meant to refer to milk level within the milk sample holding chamber that affords a reliable measurement of a milk related parameter. In one embodiment, a volume of at least 200 μl, 250 μl, 300 μl, or at least 350 μl is sufficient to allow for a reliable measurement of a milk related parameter within the specific device structure. Each possibility represents a separate embodiment of the invention.

In an embodiment of the invention, the device and its portions is a handheld device, i.e., the device is compact, portable and designed to be operated with one or both hands. The device may be small enough to be carried and used comfortably on the go. The device may be small enough to be covered by one hand. The device and specifically its monitoring compartment is small enough that it can be held and operated with one hand. In some embodiments, the device and specifically its monitoring compartment is comfortably held and operated using the fingers and as such used for the measurement.

As used herein the term “milk related parameter” refers to one or more milk parameters that can be measured by the herein system, method and device. Milk related parameters according to the preset invention include one or more of milk conductivity, milk resistivity, and milk temperature. Milk electrolytes, mainly sodium ions (Na+), are known to be effective biomarkers for secretory activation status. The content of milk sodium ions are in high correlation with milk conductivity, as shown by the inventors is PCT/IL2020/050106. Thus, the milk conductivity is an essential measurement used herein to indicate breastfeeding efficiency or milk secretory status. The milk related parameter may be displayed via a display interface of the herein system.

Non-limiting examples of electrolytes that may affect the sample conductivity as measured in the invention include sodium, potassium, chloride, calcium, magnesium and phosphate ions. In some embodiments, the main contributing electrolyte is sodium. In some embodiments, the at least one milk related parameter is the milk sample electrical resistivity or its inverse-electrical conductivity. Within the context of the invention, a low resistivity indicates a sample that is readily electrically conductive. In some embodiments, the at least one milk related parameter is electrical conductivity.

Electrical conductivity measurements or readings are used herein interchangeably to calculate a maturation score (interchangeable with the terms “milk maturation %, or MM %”). Methods of calculating maturation % were previously devised by the herein inventors and described in PCT/IL2020/050106 the content of which is incorporated herein by reference. MM % reflects the specimen maturation status as a percentage (%) within the full scale of dynamic range from initial colostrum to fully mature milk. The lower the MM % score is, the less advanced the mother is in her lactogenesis (secretory activation). MM % was designed to follow the directional progression of mothers' milk on a continuous 0% to 100% scale. MM % is inversely related to conductivity, the higher the conductivity level is, the lower the MM % is. MM % is computed based on the equation MM %=1−[(X−Xmin)/(Xmax−Xmin)]*100, where X is the raw temperature adjusted conductivity measurement. Minimal value (Xmin) and maximal value (Xmax), are the 2.5th and 97.5th percentiles extremes of the reference population dataset.

In an embodiment of the invention, the herein system provides various milk status values indicative of breastfeeding efficiency and progress, protein content, and/or breast health issues such as inflammation. The milk status values may be displayed via a display interface of the herein system. Non limiting examples of such milk status values and corresponding data outputs include breastfeeding efficiency status values including without limitation MM %, milk sample conductivity, milk sample calculated age after birth, indication for breastfeeding status as advanced or delayed relative to a corresponding reference dataset (with corresponding color and text indicators), and differences between breasts. Further milk status values include suspected breastfeeding health issues such as restricted milk flow, or inflammation. All the aforementioned information can be displayed individually for one or both breasts or as a combined value for both breasts.

As used herein, the term “milk sample age after birth” refers to the predicted age after birth calculated by the system by matching the conductivity of the measured milk or the measured MM % to corresponding values in a corresponding reference dataset. For example, when a conductivity measurement is used to determine the age of a milk sample after-birth, the system will search in a reference dataset of corresponding milk conductivities a similar conductivity measurement. The system will then determine the milk sample age after-birth associated with the identified similar conductivity measurement from the reference dataset. The calculation of milk sample age after birth incorporates combined measurements of both breasts or measurements of either breast alone.

As used herein the term “day range after birth” is interchangeable with the term “days' range” and refers to a specified interval between two points in time, measured in days after birth. This interval includes the start day, end day, and all intervening days. The term is utilized to define periods for monitoring, evaluating, or analyzing various parameters associated with breastfeeding efficiency.

In another embodiment, milk status value can be estimated Na+ levels as calculated from conductivity measurement in the sample.

As used herein the term “reference population dataset” is interchangeable with the terms “reference dataset”, “dataset derived from reference population” “benchmark” and “set of reference values” and refers to a pool of values calculated from a population dataset comprising empirical data of milk related parameters or milk values measured in a population of female subjects characterized by one or more of: lactating females at days 0-60 postpartum or more, an adequate lactation, healthy breastfeeding women, normal weight gain infant in exclusive breastfeeding with no reported breastfeeding problems. The reference dataset can be sub classified according to various parameters such as first birth, type of birth e.g., a cesarean section, preterm, and risk group for providing specific benchmarks. Different reference datasets are set for each measured status values, such as MM %, conductivity, breastfeeding progress rate, and differences between breasts. The dataset may comprise milk-related parameters or milk values measured on a daily basis after birth and optionally subdivided to daily values or day ranges after birth (optionally withing the first 21 days after birth). The population of the reference dataset may comprise more than 100 lactating female subjects, or more than 200 lactating female subjects, or more than 300 lactating female subjects, or more than 400 lactating female subjects, or more than 500 lactating female subjects, or more than 500 lactating female subjects. Each possibility represents a separate embodiment of the invention. Reference datasets may be derived from the empirical database of milk measured status values by calculating the 15^{th}, 50^{th}, 85^{th} percentiles separately for each day or day range after birth to generate a reference dataset of percentile boundaries. Milk related parameter of a certain sample taken at a certain day after birth may be compared to a reference population dataset that corresponds to the same time point. The milk related parameter of a certain sample taken at a certain day after birth may be matched to a similar milk related parameter in the reference population dataset and according to the matched measurement, a “milk sample age after birth” may be indicated for the measured milk sample.

When comparing between parameters or status values one should note that each parameter or status values measured by the herein system should be compared to a suitable corresponding reference dataset. Threshold values may be predetermined for each of the herein reference datasets such that when the measured value does not meet, i.e., exceeds, is below, or outside of a range of threshold values, a technical and/or a delayed status, and/or an alert notification may be triggered.

It should be understood that reference datasets may additionally or alternatively include data from women diagnosed with various clinical breastfeeding conditions, such as low milk supply, delayed secretory activation, primary lactation failure, insufficient glandular tissue, and infant failure to thrive. Breastfeeding health status in a subject may be detected by the herein system when the measured parameters and milk status values correspond to values of a reference dataset derived from subjects suffering from clinical breastfeeding conditions.

In some embodiments, risk groups reference dataset may be derived from pre collected dataset comprising milk status values data related to women with conditions that are known to affect successful breastfeeding including but not limited to C-section, instrumental delivery, vacuum delivery, pre-term delivery, low birth weight infant, hormonal imbalance or hormonal health conditions such as thyroid imbalance, polycystic ovaries, gestational diabetes. When a subject is identified as a member of a risk group as detailed herein, her measurements are compared to the corresponding risk group reference dataset.

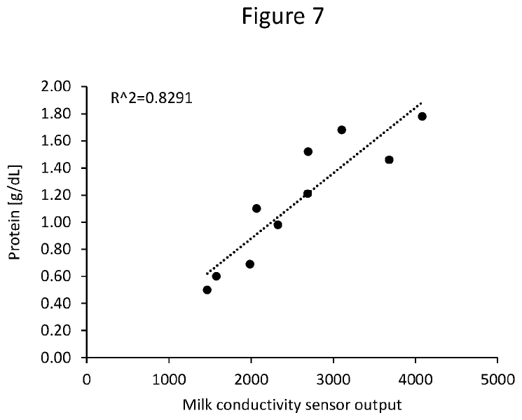

As used herein “milk protein content” refers to the calculated milk protein content in the sample. The milk protein content may be expressed in various ways, e.g., milk concentration (such as grams per deciliters), or percentage. Surprisingly, as illustrated in FIG. 7 and presented herein in table 1, the herein inventors have discovered that protein milk concentration can be reliably estimated from milk conductivity measurements. The protein content of milk is an essential indicator of its suitability for various applications, such as infant nutrition, and clinical assessments. Evaluation of milk protein content is essential for ensuring compliance with nutritional requirements and optimizing the health benefits of breast milk to premature infants. Utilizing the herein milk conductivity measurements allow to readily extrapolate the content of protein in the milk sample. This capability is significantly important as it may afford a fast robust easy to use and low-cost alternative to the typical laboratory direct milk measurements.

As used herein breastfeeding health issues include without limitation restricted milk flow, engorgement, ductal infection, mastitis, breast inflammation, breast infection, breast candida.

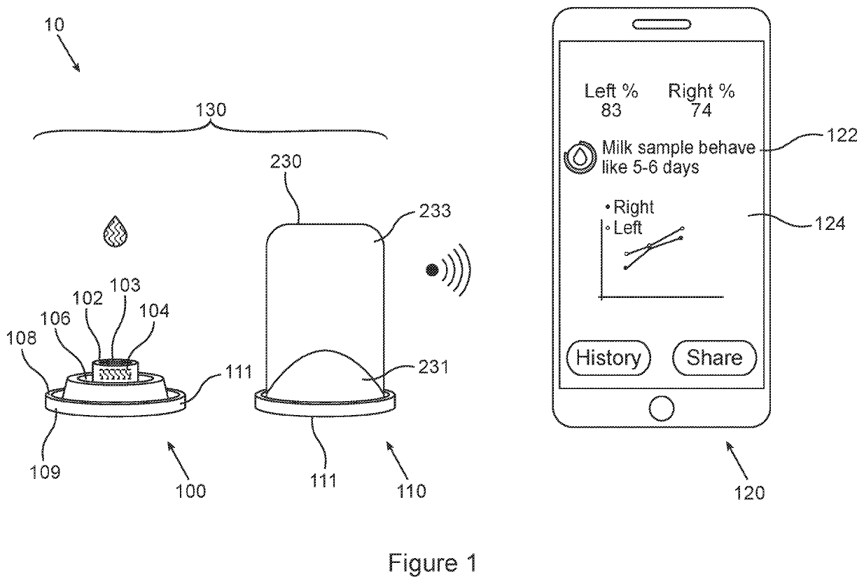

For example, FIG. 1 illustrates an exemplary milk sensing system 10 for monitoring breastfeeding efficiency of a lactating subject in accordance with an embodiment of the present invention. System 10 may include a milk sample monitoring device 130 comprising a milk sample holding chamber 100, and a monitoring compartment 110. The system 10 may further include a computing device 120.

The milk sample holding chamber 100 may include at least one chamber 102 comprising a milk sample cavity 103 for holding a sample of milk 104. The at least one chamber 102 may be configured to feature an overflow mechanism for directing milk that exceeds the predetermined maximal sample volume of the at least one chamber 102 into an overflow channel 106 for receiving the surplus milk sample 104. The overflow channel 106 may be coupled to one or more outlets 107 of the at least one sample cavity 103 for diverting excess sample out of the sample cavity 103 and into the overflow channel 106. There is optionally an elevated platform 113 on top of the milk chamber, and overflow channel 106 reside on this platform. The milk sample holding chamber 100 may comprise coupling means 108, which may comprise a walled ridge 109 on the base 111 of the milk sample holding chamber 100 for correct coupling and placement of the milk monitoring compartment 110 in relation to the at least one chamber 102.

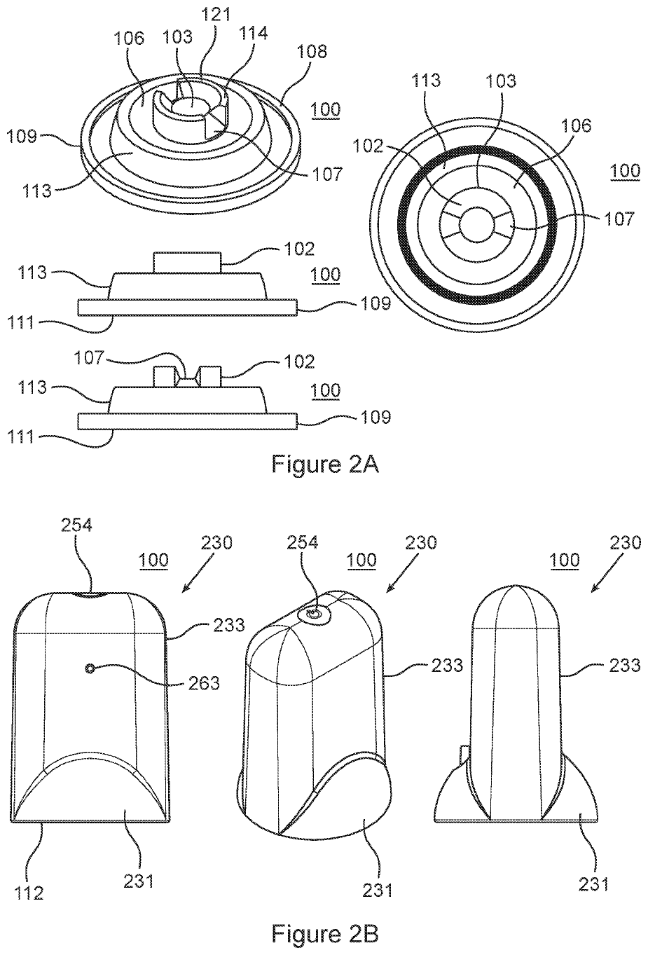

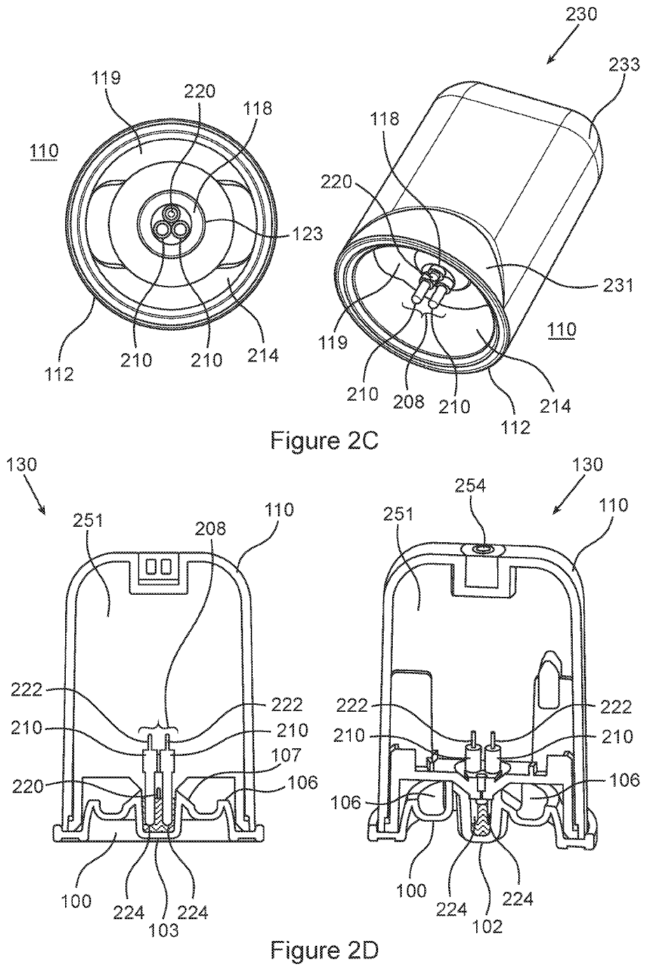

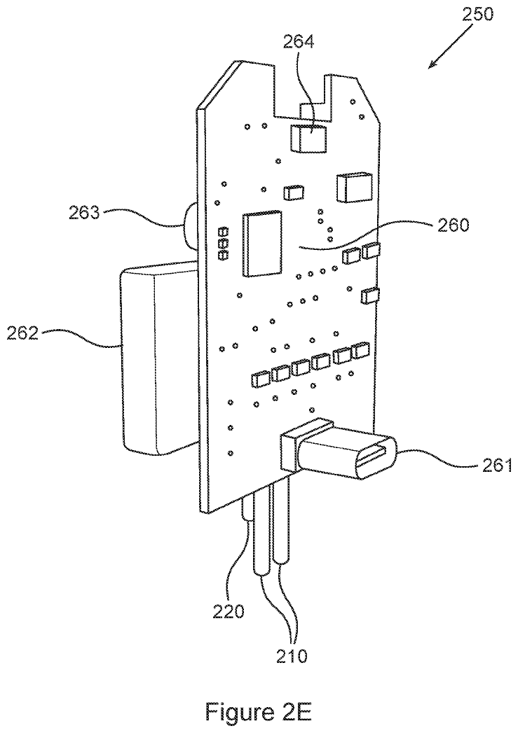

As shown in FIGS. 2C-2D, the monitoring compartment 110 may include a sensing module 208. The sensing module 208 may comprise at least one sensor 208 for sensing a milk related parameter. The at least one sensor 208 may include at least one electrode 210, such as for example two electrodes 210 for measuring conductivity of the milk. The at least one sensor 208 may comprise at least one sensor for measuring temperature 220. Monitoring compartment 110 may include a processing unit 250, for processing data relating to the milk related parameter measurement. The processing unit 250 is in communication with the at least one sensor 208 and is also in communication with a computing device 120. The computing device 120 may be a remote computing device 120.

Returning to FIG. 1 the milk monitoring compartment 110 configured to correspond for optimal coupling and fit with the milk sample holding chamber 100 for milk measurement, such that the sensing module 208 is correctly and sufficiently accommodated within the milk sample cavity 103 holding the human milk sample 104 for sensing and measurement of milk related parameters. The monitoring compartment 110 comprises a shell housing 230 comprising a shell base 231 and a shell body 233. The shell housing 230 encapsulating the processing unit 230 and at least the proximal portion of sensing module 208 within the shell housing cavity 251. The sensing module 208 is positioned within the shell housing 230 such that at least the sensing portion thereof extends down vertically from the body 233 of the shell housing 230 towards the open shell base 231. The shell base 231 comprises a peripheral wall enclosing a shell base cavity 214 (shown in FIG. 2C) configured to accommodate at least the internal portion of the milk sample holding chamber 100 such that the at least one sensor (210,220) of the sensing module 208 is positioned within the at least one milk sample chamber cavity 103 for contact with the sample 104. In some embodiments, the shell base opening 112 of the shell base 231 is coupled to the sample holding chamber by corresponding coupling means 109 on the milk sample holding chamber 100, such that shell base 231 opening 112 of the monitoring compartment 110 is surrounded by coupling means 109. Coupling of the monitoring compartment 110 to the milk sample holding chamber 100 may be by any suitable attachment means. In one embodiment the milk sample holding chamber 100 comprises a base 111 with a walled ridge 109 configured to receive the open shell base 112 of the monitoring compartment 110 when placed there. In one embodiment, coupling comprises placing the milk sample holding chamber 100 on a flat surface and placing on top of the milk sample holding chamber 100 the monitoring compartment 110 such that the walled ridge 109 surrounds the perimeter of shell base opening 112 and center of gravity and structure of the device maintains the coupling. The milk sensing system 10 may be in communication with a remote computing device 120, such as, but not limited to as in this implementation a smart phone 120, with an internet gateway. The smart phone 120 can display data 122 received from the monitoring compartment 110.

Referring to FIG. 2 for a more detailed description of the milk sample monitoring device 130. In one embodiment, the monitoring compartment 110 comprises at least one sensing module 208 for measuring and sensing at least one parameter relating to the milk sample 104. The at least one sensing module 208 is in communication with the processing unit 250 (shown in FIG. 2E). The at least one sensing module 208 may comprise at least one electrode set, such as conductivity electrodes 210 in communication with the processing unit 250 (shown in FIG. 2E). The at least one sensing module 208 include an electrode set for measuring conductivity, such as two electrodes 210, which can be positioned so that when the two parts of the milk sample monitoring device 130, the milk sample holding chamber 100 and the monitoring compartment 110 are connected together, the distal end 224 of sensing module 208 is positioned within the cavity 103 of at least one milk chamber 102 (shown in FIG. 2D). The sensing portion of the conductivity electrodes 210 is in sufficient contact with a pre-defined milk volume as to receive a reliable conductivity reading. The conductivity electrodes (herein termed also conductivity sensor) 210 can then provide raw conductivity or resistivity data to the processing unit 250. The proximal end 222 of the sensing module 208 is connected to the processing unit 250.

FIG. 2D shows the milk sample holding chamber 100 and the monitoring compartment 110 coupled together such that the shell base 231 of the milk monitoring compartment 110 forms a dome like structure over the milk sample holding chamber 100. The at least one sensing module 208 such as an electrode set comprising conductivity sensor electrodes 210 and the at least one temperature sensor 220 are shown extending out of the shell base 231 and into the sample cavity 103 of the sample holding chamber 100 when the monitoring compartment 110 is placed on top of the sample holding chamber 100 for analysis of the milk sample.

Once the processing unit 250 receives the raw conductivity data of the milk sample 104, the processing unit 250 processes the raw data. In particular, the processing unit 250 can correct the conductivity value from a raw conductivity value as measured by the conductivity sensor 210 according to the milk sample temperature measured by the temperature sensor 220. The conductivity of breast milk is influenced by the temperature of the sample at measurement. Breast milk is typically extracted from the breast at body temperature (36-37° C.) and gradually reaches ambient temperature, causing a fluctuation in the milk conductance. For a correct evaluation of the milk maturation % of the milk extracted for measurement, the value of the conductivity of the milk is adjusted/corrected by the system of the present invention 10 by taking the conductivity reading and the measured temperature and calculating a normalized conductivity (for 25° degrees Celsius). This allows for reliable calculation of % maturation and comparison between different readings taken at different temperatures. This is also important for enabling a reliable comparison of the measured milk related parameter or milk status value to a reference dataset according to sample database achieved in comparable conditions.

The at least one monitoring compartment 110 may include a wireless transmitter 260 for transmitting data as shown in FIG. 2E to the computing device 120 via local wireless communications, e.g., Bluetooth, or a local internet gateway. The processing unit 250 can then provide the data to the wireless transmitter 260.

The system for monitoring breastfeeding efficiency 10 includes an electric circuit communication between the conductivity sensors 210 and the processing unit 250 for sensing conductivity related data in the milk sample 104 in the milk sample chamber central cavity 103. Since even a small change in milk sample amount can affect reading reliability, the monitoring compartment 110 may include a second electric circuit generating additional data output regarding the sufficient milk sample level, such as identifying an adequate volume and/or height/level of milk sample 104 in the milk sample cavity 103 for reliable reading of the milk sample 104. The at least one sensing module 208 may further include a volume sufficiency sensor 211 which may be part or separate from a temperature sensor 220. In one exemplary embodiment, temperature sensor 220 is encapsulated within a conductive casing 211 and placed at a designated position relative to the conductivity electrode set 210. In some embodiments, the conductive casing 211 is electrically connected to one of the conductivity electrodes 210 enabling the generation of an electric signal that is achieved when sample is simultaneously in touch with the conductivity electrode 210 and the casing 211 of the temperature sensor 220 indicating a sufficient sample height/level for reliable conductivity measurement. In some embodiments, the temperature sensor 220 is positioned between the conductivity electrodes 210 but not on the same plane. For example, temperature sensor 220 may be positioned on a different plane, and in different length, and together with the electrodes 210, the sensing module may form a triangular structure. This specific design affords a compact sensing arrangement that can fit the small size of the milk chamber 102 affording a reliable measurement while maintaining a small volume of sample. The processing unit 250 receives this output and generates a notification alert according to whether the milk sample level is sufficient to be reliably read by the system (milk sample volume sufficiency). The components of the milk sample holding chamber 100 are configured to hold a volume of milk sample, which has a minimal volume for enabling sensing of the milk sample 104 and a maximum volume for proper working of the system 10. In some embodiments, the cavity 103 of the milk chamber 102 is configured to accommodate a milk sample volume of one drop of milk, two drops of milk, three drops of milk, four drops of milk, five drops of milk, six drops of milk, seven drops of milk, eight drops of milk, nine drops of milk, or ten drops of milk. Each possibility represents a separate embodiment of the invention.

The processing unit 250 can process a plurality of milk related parameters and calculate and identify status values related to the data received from the monitoring compartment 110 about the milk sample 104. As described above, the milk related parameters can include, but are not limited to conductivity of the sample, resistance of the sample, temperature of the sample, sample sufficiency signal and other physical properties relating to the milk sample 104 reading. In one embodiment, the processing unit 250 can also identify at least one of the following: indicate when the monitoring compartment 110 is running low on battery, when the Bluetooth is disconnected, or other functions that relate to the operation of the present invention.

When processing unit 250 detects a problem within system 10 (e.g., not enough sample, low battery, poor signal strength, etc.) it can provide an indication of the problem. For example, the monitoring compartment 110 can light an alarm in the monitoring compartment 110 and display a notification alert on the remote computing device 120.

The wireless transmitter 260 and the processing unit 250 can be located on a common circuit board. In some cases, processing the data at the processing unit 250 before transmitting the data with the wireless transmitter 260 can improve the data and lower the error rate associated with the data. In particular, processing a milk sample volume alert by the processing unit 250 when the milk sample level in the milk chamber 102 is insufficient (for example <~250 μl) for generating a reliable reading can trigger an immediate user's notification alert to correct milk sample volume for a rescan. An insufficient milk sample for reading by the system 10 is a predominant user technical error with significant implications on result accuracy and therefore should be avoided, especially when the milk sample 104 is collected in the first days after birth when colostrum is produced in very small volumes.

Once the data has been processed and transmitted to a receiving second processing unit, such as computing device 120, the remote computing device 120 can further analyze the data. The remote computing device 120 includes an application software program of the present invention for analyzing and displaying the received milk related parameter and/or milk status values. In particular, the remote computing device 120 can process the data to identify trends within the conductivity data that can be used to define the secretory activation level of the lactating subject and/or to calculate and present the milk maturation % parameter and any other status values of the milk. For example, the remote computing device 120 can identify that the conductivity measurement of the milk sample exceeds a specific threshold value or is out of a specific range of values relative to a set of reference dataset, defined for each day or day range after birth (also referred to as baby age in days).

If the computing device 120 detects a milk status value or if it detects a technical issue (e.g. not enough milk sample, low battery, etc.), or a potential breast health issue such as breast inflammation, the remote computing device 120 can provide an indication regarding the identified issue, or of the technical problem to the user. For example, the remote computing device 120 can display a color indication, and/or provide a text notification to a user, and/or provide a vocal notification to a user.

In one embodiment, the remote computing device (e.g., smart phone) 120 can display the interpretations of the various data that it is receiving and has analyzed. For example, the smart phone 120 can show the sensed milk sample result for milk from one breast or from each breast side, or it can provide a combined result. As breastmilk is a unique bodily fluid in the sense that it is generated in two partly independent organ units (in a feedback loop with the suckling baby, and frequency and effectivity of breast feeding or pumping or direct stimulation from that side, and also other local physiological differences in the tissue) it is preferable to monitor both sides for reliably identifying breastfeeding related issues. The visual output can be milk sample values and/or milk related parameter as received from the monitoring compartment 110 or computed parameters based on the received conductivity values. In some embodiments, the results are presented as milk maturation percentages. The display interface 124 of the computing device 120, at least in one embodiment, presents separate data for each breast, the left breast and the right breast. In some embodiments, the displayed results can be assigned with a color indicative of sample status relative to a set of reference dataset values. The computing device 120 can configure matched milk-age notifications generated by matching a sensed milk related value parameter to the dataset of reference values, determining a range of MM % that corresponds to a certain percentile (e.g., 25-75 percentile) of the database of the corresponding day or days range after birth. In at least some embodiments, the display interface 124 can show a separate graph for each breast, tracking the computed milk MM % calculated from milk conductivity as recorded over time for each breast. The graph can include an indication of reference dataset values to illustrate to the user the comparison between her results and a breastfeeding behavior in typical (i.e., normal) reference population. Additionally, the computing device 120 can display historical information relating to the received data. For example, the computing device 120 can display a log of all the measurements by date, days from birth, results, and color indications.

In some embodiments, the system can 10 further independently generate an estimated amount of protein in the milk sample 104.

The computing device 120 can further analyze the data received compared to historical data from the same woman to generate additional breastfeeding status insights and technical alerts. For example, in one embodiment of the invention, a conductivity or MM % value of a tested milk sample may be compared to at least one previous reading(s) of the same woman, providing evaluation regarding the breastfeeding progress over time. The breastfeeding progress rate may be evaluated by calculating a derivative of a MM % line graph or calculating the difference and comparing therebetween. The evaluated breastfeeding progress rate may be compared to corresponding reference dataset of milk related parameter progress rates over the corresponding day range of milk related parameters. This also can be used for setting personalized progress targets and expected daily goals by choosing the goal according to the relation to the corresponding reference dataset. Progress of the MM % reflects the progress of breastfeeding establishment and increased milk supply. In other embodiments, when progress rate exceeds a threshold of a reference dataset, it may alert the user that this may represent a technical error and there is a need to perform a repeated measurement.

Computing device 120 can further analyze the received data to generate breast health related visual outputs. For example, comparison of the received conductivity values of the two breasts can provide information regarding breast side preference, and imbalance between the breasts regarding milk production. Combining the measured milk related parameter data with a reported breast pain indication(s), and breast differences in the conductivity data can alert early trends that may be linked to breast health issue such as inflammation.

In general, the computing device 120 can utilize the received information to display via display interface 124 a variety of useful data relating to lactation, breast health, human milk properties, infant nutrition, infant health and feeding health of the infant, all of which are contemplated by the present invention.

Additionally, the remote computing device 120 further comprises a module in which a user can record breastfeeding related information which can be used for generating additional user interface data. For example, daily breastfeeding sessions can be reported by the mother, and specific guidance can be generated by the system 10 when a delayed breastfeeding status is identified (i.e., based on the milk conductivity data) along with reporting a behavior pattern (e.g., sparse breastfeeding routine). In additional non-limiting examples, breastfeeding confidence scores, breastfeeding pain, baby weight gain, or sleep patterns can be recorded and computed together with conductivity data for generating and adjusting lactation and infant related data.

Additionally, the remote computing device 120 can access a historical record of recorded milk related parameters of the same user. For example, a mother can access a historical record of human milk conductivity data received from the monitoring compartment 110, or any recorded value based on the measured data, such as MM % and provide the record to a caregiver e.g., a lactation consultant. The historical data can also include additional health records logged by the mother. The accessed historical record can be stored by the remote computing device 120, or some other web based storage cache or database.

The wireless transmitter 260 can be an internet gateway that transmits data wirelessly to a secure web portal. The transmitted data can then be accessed through a password-protected associated webpage. Additionally, in one embodiment, a remote computing device 120 can access a historical record of data recorded by several independent systems. For example, in one embodiment of the invention, a caregiver, such as a lactation consultant, can access data recorded from several users, each from an independent milk sample monitoring device 130 for performing milk measurements and for using said milk measurements by an independent computing device such as a smart phone 120 for data receiving, recording, processing and display. In one embodiment, an additional computing device can generate patient status notifications and alerts based on a milk conductivity or a milk maturation computed parameter, compared to predefined set of reference values. In one embodiment, for example, a milk related parameter indicative of a breastfeeding status of low milk supply or delayed secretory activation breastfeeding status relative to a corresponding reference dataset can be presented to a lactation consultant of a breastfeeding mother with slow weight baby's gain or failure to thrive, as reported by the lactation consultant. For example, the present invention can be used for prioritizing care and breastfeeding support and identifying patients at higher risk for insufficient milk supply.

In one non-limiting embodiment, the same system 10 or parts thereof can be used by multiple users. Each user may be identified for proper collection and analysis of the data relating to the user's breastmilk.

In one non-limiting embodiment, the same system 10 or parts thereof may be used for analyzing lactation of more than one infant, such as twins, triplets and other multiple births or different aged siblings receiving human milk from the same woman. Each infant may be distinctly identified by the system 10, and optionally outputs associated with milk related parameters thereof could be analyzed relative to reference datasets derived from empirical data previously collected for this specific subgroup of multiple birth.

In one non-limiting embodiment, the same system 10 or parts thereof may be used for analyzing lactation of a preterm infant and optionally comparing to a reference dataset calculated as detailed above but based on empirical data for this specific subgroup of preterm infants.

In one non-limiting embodiment, the same system 10 or parts thereof may be used in a NICU for analyzing lactation of multiple preterm infants, comparing to a reference dataset calculated as detailed above but based on empirical data for this specific sub group of preterm infants, and computing estimated milk protein content (expressed in % or concentration) for directing targeted fortification of the infant.

One will understand that the embodiments described above are only exemplary and that a system of the present invention can comprise a fewer number or a greater number of components. For example, the present invention can comprise only the milk sample holding chamber 100 and monitoring compartment 110, where there can be one processing unit 250 within the monitoring compartment 110 and the monitoring compartment 110 can comprise a display unit (not shown) or a visual interface for displaying an alarm output. Similarly, in another embodiment, the present invention can comprise a different receiving station unit where data can be received from the monitoring compartment 110, processed, and generate a visual indication for an alarm output. Additionally, the present invention can comprise only the monitoring compartment 110 and optionally also the computing device 120 where the milk sample chamber can be integrated within the monitoring compartment 110, or the monitoring compartment can be designed to take a pre-defined volume for the measurement of the milk.

FIG. 2A-2E illustrate a milk sensing system 10 including a milk monitoring compartment 110 and a milk sample holding chamber 100 that are configured to perform accurate conductivity measurements of pre-defined small volumes of milk. In particular, FIGS. 2A-2C depict an embodiment of the present invention that comprises a monitoring compartment 110 with an electrode set 210 and a milk sample chamber 102 fit to hold a sample volume 104 to be in direct contact with the electrodes 210 and the temperature sensor 220.

FIG. 2D illustrates an embodiment of the milk monitoring compartment 110 when coupled to milk sample holding chamber 100, for performing a human milk measurement. FIG. 2E illustrates the processing unit 250 of the monitoring compartment 110 including the electrical board 260, the set of sensor electrodes 210, the temperature sensor 220 which is also part of the milk level/volume sufficiency 211 electric circuit, and other elements such as a wireless transmitter 260 for data transfer.

In one embodiment, the configured cavity 103 of the milk sample chamber 102 is encapsulated between the monitoring compartment 110 and the milk sample holding chamber 100 and is designed to hold a certain very low volume of milk. The novel configuration of the system of the present invention which enables analysis of a very small volume of milk is important for implementation of the system for measuring colostrum, the initial milk at the first days after birth which is produced in small quantities. A low volume milk sample for sample reading is also important for clinical implementations in cases of low milk supply or slow weight gain in the baby where milk production is limited. As a non-limiting example, the milk sample holding chamber 100 and the enclosed cavity 103 of the milk chamber 102 with the sensor is designed to hold about 250 microliter of milk sample. In other embodiments sample cavity 103 of the milk chamber can be designed to hold smaller volumes of about 5-50 microliters. In other embodiments the sample cavity 103 of the milk chamber 102 can be designed to hold volumes of about 50-150 microliters. In other embodiments, sample cavity 103 of the milk chamber 102 can be designed to hold about 150-250 microliters. In other embodiments, sample cavity 103 of the milk chamber 102 can be designed to hold about 250-500 microliters. In other embodiments sample cavity 103 of the milk chamber 102 can be designed to hold about 500-3000 microliters. In some situations, to help improve the quality of the sensor reading, the milk sample holding chamber 100 can allow for larger milk sample volumes. This facilitates including more representative milk samples, and reducing the electric interference created by the milk cell walls. In non-limiting examples, the milk sample chamber 102 can be designed to hold different volumes of from between 150 to about 3000 microliters. In some embodiments, the sample holding chamber 102 is configured to hold a volume, which must be above a minimal volume and below a maximal volume (the ranges described hereinabove) for the system to be able to reliably monitor and read the milk sample.

In one embodiment, the monitoring compartment 110 features a unique electrode centered arrangement and a unique mechanical enclosure structured to enclose the milk sample holding chamber 100 creating a closed cell where the electrode set 210 and temperature sensor 220 are substantially centrally embedded within a defined volume of milk sample 104, which generates a stable reading environment for reading milk related data, which prevents inaccuracy of readings due to small sample related interferences. The enclosure is formed when monitoring compartment 110 is coupled to the milk sample holding chamber 100 and is thus defined by the walls 114 of the sample cavity 103 and the top roof 119 and/or a protruding bulge 118 of the shell base 110. In an exemplary embodiment, the enclosure formed may be configured to hold a milk sample volume of up to about 250 μl.

FIG. 2D illustrates a depiction of a cross section of the encapsulated cell created between the monitoring compartment 110 (upper structure) and the milk holding chamber 100 (lower part), designed to hold a minute pre-defined fixed volume of liquid 104, with the sensing module 208 centralized within, for accommodation of the sensors 210 within the milk chamber sample cavity 103.

In one embodiment as previously described the monitoring compartment 110 includes at least one conductivity sensor 210 and at least one temperature sensor 220 in a designated layout, allowing adjustment or correction of the conductivity measurement according to the specific sample temperature, such as a cold (e.g. pre frozen or refrigerated milk) or warm sample (freshly expressed) or a sample temperature varied by the ambient temperature at the time of measurement. For example, in one embodiment of the invention, the monitoring compartment 110 includes a set of two conductivity electrodes 210 and one temperature sensor 220, the temperature sensor 220 placed between the two conductivity measuring electrodes 210. The configuration of the temperature sensor 220 is with an offset and with a shorter tip for less deep immersion into the sample cavity 103 of the milk chamber 102 than the conductivity measuring electrodes 210, allowing for measuring and reading the milk sample temperature without interfering with the conductivity measurement reading. This particular arrangement of sensors and milk chamber 102 advantageously affords reliable and accurate milk conductivity measurements with minimal deviations and reading errors. Further, the herein configuration affords the above attributes even with minute volumes of colostrum, providing for the first time a solution to the problem of accurately measuring milk conductivity in early stages of breastfeeding.

A processing unit 250 within the monitoring compartment 110 may execute a computational correction to the raw conductivity measurement according to the measured sample temperature, to predict milk conductivity of the sample at a fixed temperature. This allows for reliable further algorithm steps as calculation of % maturation in a way that allows reliable comparison between different readings done at different ambient temperatures, and comparing between reads to reference values set according to dataset that was achieved and normalized in a unified manner.

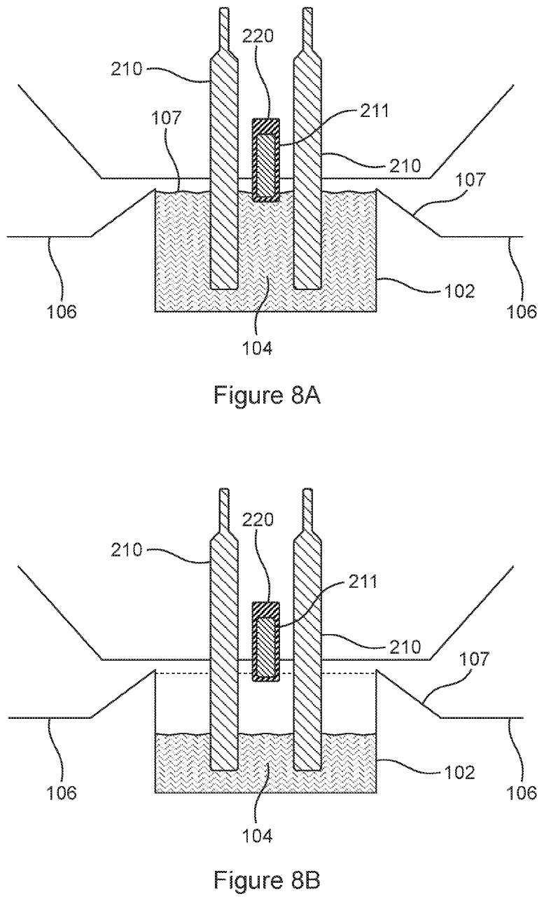

In one embodiment, the milk monitoring compartment 110, and/or computing device 120 is configured to provide an alert when the milk sample volume is not sufficient for reliable measurement as shown in FIG. 8A and FIG. 8B. In one embodiment, the monitoring compartment 110 affords a milk sample level sufficiency indication, where a conductive material casing 211 in touch with the milk sample surface 107 closes an electric circuit, generating an output regarding sufficient milk sample level. For example, the temperature sensor 220 casing 211 is made of a conductive material that is embedded in a specific electric circuit in the board and may be positioned at a specific level within the sample cavity 103 when the milk holding chamber 100 and the milk monitoring compartment 110 are coupled together. In a non-limiting example, when the milk volume is sufficient as shown in FIG. 8A, the sample 104 touches the conductive casing 211 of the temperature sensor 220 closing the circuit with one of the two conductivity sensing electrodes 210, generating a milk level signal, indicative that the milk sample level 107 is sufficient for a reliable reading by the system 10. When the sample volume is lower than the minimal required sample volume as shown in FIG. 8B, the surface of the sample level is below the conductive material casing 211 of the temperature sensor 220 and the sample 104 does not touch the conductive casing 211 of the temperature sensor 220 and an electronic circuit is not closed, which provides an alert that the milk sample volume is insufficient for an accurate measurement by the system 10 of the present invention.

In one embodiment, the milk sample holding chamber 100 is designed to hold a specific milk volume when coupled to the monitoring device's shell base 231. The herein coupling is configured to remove excess sample for generating accurate readings as described hereinabove. For example, FIGS. 2C and 2D. show a milk sample holding chamber 100 configured of a set of connected walled cavities generating a tunnel system of at least one channel for holding a sample in its cavity and at least one overflow channel 106. In this example, milk is collected to the inner cavity 103 of the milk sample chamber 100, and when placing the monitoring compartment 110 on top of the milk sample chamber 100 for coupling of the monitoring compartment 110 and the milk sample holding chamber 100 as described hereinabove, the at least electrode set 210, the temperature sensor 220 and the shell base 231 of the monitoring compartment 110 push the liquid volume 104 within the inner cavity 103, so that an excess of sample volume is pushed to an overflow spaced apart linked channel 106 allowing the sample volume to be adjusted to the fixed volume required for a reliable measurement.

In one embodiment, there are two milk sample chambers 100, each configured to collect separately a milk sample from each breast, allowing identification of different trends in each breast, and also avoiding the need to clean the chamber between a second measurement of milk from the other breast. In one embodiment, the milk sample chamber 100 is designed as two connected sample chambers, each with a breast side identification mark. In one embodiment, the milk sample chamber 100 is designed to have a cap (not shown) allowing the moving of a filled sample chamber from one site to another site.

In one embodiment, the milk sample holding chamber 100 is a disposable part. In another embodiment, the milk sample holding chamber 100 is a reusable part. In particular, the milk sample holding chamber 100 can be comprised of silicone or plastic. One will understand that the ability to dispose the milk sample holding chamber 100 after use provides several benefits. For example, single use parts reduce the hassle of cleaning parts, and avoid technical invalid results due to any residual prior sample or residual washing residue. Additionally, this allows easy replacement of parts of the system, such as but not limited to missing parts and broken parts. This can also allow for use for different women with one system by a use of clinician.

The sensing surface of the monitoring compartment 110, which is in contact with sample liquid 104, is configured to be water resistant. For example, the electrode set 210 and the conductive casing of the temperature sensor 220 are designed to be of a non-corrosive conductive material. Additionally, the shell housing 230 of the monitoring compartment 110 is of water resistant material (plastic or silicon) and may be configured as one part with the shell housing 230 material over-molded on electrodes.

In one embodiment, the milk sample chamber 100 and the monitoring compartment 110 parts are configured rounded and allow simple use of the system 10 parts by the mother. Simple connection by design between the two parts 100, and 110 have the benefits of consistent error proof sample cavity 103 enclosure generated by the coupling between the two parts 110, and 110 and correct and accurate placement of the electrodes 208 within the milk sample holding chamber 100.

However, one will understand that the design of the parts of the milk sample holding chamber 100 and the coupled monitoring compartment 110 may not be round and may be of any suitable shape and may contain two cavities for right and left breast samples or any suitable number of cavities for holding samples and the analysis thereof.

While the above-described implementations may describe embodiments with two electrodes 210 and one temperature sensor 220, the present invention can also be practiced with a variety of different sensing module unit 208 configurations. For example, more than two electrodes 210 can be utilized.

In one embodiment of the invention, the monitoring compartment comprises a set of two parallel cylindrical electrodes 210 which are connected in series with a resistor (not shown). For example, a depiction of an implementation of the conductivity sensor 210 comprises applying a bipolar pulse to an electrical conductivity (EC) cell which is connected in series with a resistor (not shown) and measuring the voltage on the node between the EC and the resistor. In one embodiment, for calculating conductivity, the bipolar pulse is run with a series of EC measurements between the poles at a set of at least two time points allowing for determining a stable read and using the average reading between the two or more readings or only the second or last reading as the raw conductivity for further analysis.

FIG. 2E depicts an implementation of a portion of the monitoring compartment 110 of the present invention. In particular FIG. 2E depicts the processing unit 250. The processing unit 250 can include means for processing conductivity data on site. Specifically, the processing unit 250 can include the necessary means for processing the raw voltage or current data and relaying the data to the computing device (e.g., a remote phone device) 120 or other types of wireless transceivers.

For example, the processing unit 250 can convert the raw data into a format that can be broadcast through a particular wireless transmitter (e.g., Bluetooth, etc.) 260. One will understand, however, that various transmission formats are known in the art and any type and combination of known transmission formats can be used and remain within the scope of the present invention.

Additionally, processing unit 250 can receive a raw electrical signal from the EC cell and apply computational algorithms and calculations. As processing unit 250 processes the received raw data, the processing unit can determine at least a conductivity measurement of a milk sample. In one embodiment, the processing unit 250 also collects the raw data generated by the temperature sensor 220 and uses the measured value for correction calculation to predict sample conductivity at 25 degrees Celsius. In one or more embodiments, the processing unit 250 uses a third raw electrical output from the electrical circuit featuring the temperature sensor (thermometer) 220 as described hereinabove to generate an alert when the milk sample volume is not enough for accurate measurement.

Herein are some non-limiting examples of computational processes performed by the processing unit 250, which may be implemented by the present invention.

In one embodiment of the invention, the processing unit 250 may apply an algorithm to run a bipolar pulse with a series of EC measurements between the poles, at a set of at least two time points, and apply a computational equation for calculating conductivity based on the measured parameters. In one embodiment the voltage measurement is in the first half pulse, and the first time point is at t=0.

The processing unit 250 may further execute a computational calculation based on the specific sample EC cell constant. The EC cell constant is defined by cell properties such as distance between electrode pair, electrode surface in contact with the sample, and the milk sample volume encapsulated in the sample cavity 103 disposed between the monitoring compartment 110 and the milk sample holding chamber 100.

In one embodiment, the EC constant is adjusted by a standard curve linear correction factor empirically defined by running a testing set of known standard solutions with known conductivity, in the designated milk cell chamber 100 and using the monitoring compartment 110.

Additionally, processing unit 250 within the monitoring compartment 110 may apply an algorithm on the raw measured milk related parameter for increased accuracy. For example, in one embodiment, the processing unit 250 may apply an algorithm to adjust the raw conductivity measurement according to the milk sample temperature measured, to predict milk conductivity in a fixed temperature which can be any temperature determined for normalizing the herein measured conductivity e.g., ambient temperature. An additional example may include applying an algorithm to apply a series of measuring cycles, in a defined time frame, to obtain a reliable conductivity reading.

In one embodiment, the processing unit 250 can also alert when there is not enough milk sample as described hereinabove. In particular, the processing unit 250 can trigger an alert when there is no sufficient current measurement in the second electrical circuit generated by the conductive casing of the temperature sensor 220 and one or more of the conductivity electrodes 210.

In one embodiment, a processing unit 250 can also apply an algorithm and trigger an alert when a technical error is detected. For example, technical error comprises conductivity measurement that is too low or too high compared to an extreme set of pre-determined values derived from the full range of reference milk samples dataset, e.g., minimum and maximum values of the full physiologically relevant range of diverse tested milk samples. Erroneous conductivity measurements can be caused by various human errors or environmental factors including, but not limited to, air bubbles, water dilution, detergent or ointment leftovers or contaminations, improper mechanical use etc. or technical and mechanical malfunction in the device itself including electrode displacement, faulty electrical wiring, or faulty mechanical structure etc. A technical error further includes additional technical issues associated with milk sample monitoring device 130. For example, battery that is running low. The processing unit 250 may apply an algorithm to ensure that there is enough battery for reliable measurement and alert a user when battery charging is required.

In one embodiment, the processing unit 250 can trigger an alert, which may be displayed on the milk sensing device, e.g. by the color of the LED light 263 or the LED light being lit, for immediate notification. In addition, the processing unit 250 can transmit the data with the wireless transmitter 260, to a remote computing device 120, e.g., smartphone 120 or a server for further data analysis and display.

In addition, the above-described implementations are not limited to being practiced by the monitoring compartment unit 110 and the processing unit 250. For example, in one embodiment, the remote computing device 120 can apply the computational calculations for conductivity or adjust the conductivity. In at least one example, a combination of both the sensing device 110 and the remote computing device 120, are applied for increasing the accuracy of the milk related parameter measurements.

In addition to the processing unit 250, the monitoring compartment 110 can include at least one power source 262, such as a battery 262, that can power the monitoring compartment 110. The battery 262 can be removable and/or rechargeable. In one embodiment, the monitoring compartment 110 can also include a visual indicator that indicates when the battery is low on power and needs recharging and or when the battery is sufficiently charged.

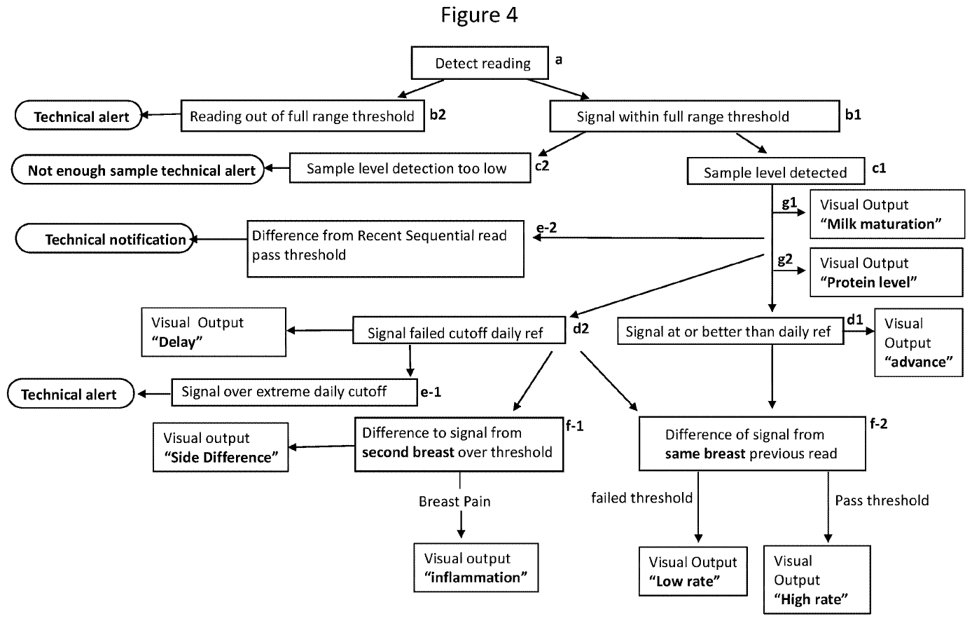

In one embodiment, the monitoring compartment 110 can also detect insufficient sample quantity that may introduce inaccuracy to the conductivity measurement. For example, a deviation that can be as small as one drop (30 microliter) below the requirement, can impact the conductivity and introduce an error of over 5%. Detecting the insufficient sample volume enables the processing unit 250 to trigger a technical alert indicating an insufficient milk sample level within the milk sample holding chamber 100. In such cases where the milk volume is too low, the conductivity may be regarded by the algorithm to be valid, relative to the references, but due to insufficient milk sample quantity, a technical alert is issued (see FIG. 4 step c). As such, instead of providing a visual output, which is likely to be false, the system notifies the computing module to generate a technical alarm and instructs the user to repeat the measurement for a more precise and reliable read.

In one embodiment, the sensing device 110 can comprise various LED lights 263 for added clarity for the user, alerting technical errors such as insufficient milk sample quantity or alerting assistive operational status wireless transmitter e.g, device charging, battery sufficiently charged, device connected to Bluetooth, device performing a scan, device Bluetooth discoverable but not linked.

Additionally, monitoring compartment 110 may include the necessary transmission components to communicate with the remote computing device 120 e.g., via Bluetooth.

The monitoring compartment 110 can also include at least one physical communications port 261. The at least one communications port 261 can be used for recharging the battery 262 within the monitoring compartment 110, communicating data to the computing device 120, updating software within the monitoring compartment 110, or some other known function.

In one embodiment, the monitoring compartment 110 can be activated by the remote computing device 120, such as a smartphone application. Additionally, in one embodiment, the monitoring compartment 110 can be activated by an on/off button on the monitoring compartment, which triggers a processing protocol, that triggers Bluetooth communication with the remote smartphone application.

FIG. 2B depicts an implementation of the monitoring compartment 110 encapsulation, comprising a plastic shell housing 230 comprising a bottom rounded protruding shell base 231 designed to fit the milk sample holding chamber 100 as depicted in FIG. 2A, for functionality, ease of use and aesthetic appeal. The shell housing 230 can be designed to allow evaluating the milk sample 104 when the milk sample holding chamber 100 is placed on a flat surface and the monitoring compartment 110 is placed on top of it. Milk sample holding chamber 100 may be used for measuring both breasts, or a separate milk sample holding chamber 100 may be used for each breast. Moreover, there may be a LED-light guide and display 263 on shell housing 230. The display may be configured to be easily seen by a user and for receiving important technical notifications and error alerts defined by the LED color, such as, but not limited, to verification of Bluetooth connection, milk scan status (e.g., verification of performing a milk scan), low milk volume alert and/or low battery alert. The button 254 on the shell housing 230 of the sensing device 110 may be of various types, such as the design shown in the drawing. Shell housing 230 includes a bottom rounded and optionally protruded shell base 231 connected to shell body 233 which together may form a bell-like structure. The shell body 233 allows for a convenient gripping by a user's fingers. As used herein the shell features various structures including a rounded, an oval, a square, or a rectangle shape. Each possibility represents a separate embodiment of the invention. The shell base 231 may comprise a top internal roof 119 that divides the shell housing of the monitoring compartment such that the shell body 233 accommodates the processing unit 250 and a proximal portion of the sensing module 208, and shell base 231 accommodates the distal portion of the sensing module that contacts the sample. As depicted in FIG. 2C, optionally the shell roof 119 comprises a protruding bulge 118 from which the at least one sensor vertically extends downwardly. The external circumference 123 of bulge 118 may fit in size to the internal opened circumference 121 of chamber 102 and the slope of the bulge 118 fits the slopes of the milk sample cavity walls such that it fits to close the opening of the milk cavity and pushes excess sample through the outlets 107 to the channel 106.

One will understand that the unique milk sensing components can be integrated with other breastfeeding aids known in the art, and still be part of the current invention. For example, monitoring compartment 110 can be positioned within or coupled to a breast pump device. The pump device may feature a pump mechanism, a “breast shield” that wraps around the breast and a container to hold the human milk, and the pump force is generated mechanically or by engine. The breast pump part can include the necessary electrical conductivity cell components, including at least the electrode set 210, to generate a milk conductivity reading, which in a non-limiting example, is measured from the human milk flow through the breast shield, or from the human milk held in the container, with a mechanical design to hold the defined milk volume for the reading. The raw electrical signals can then be processed by a processing unit that can be integrated with the processing unit of the pump, to generate a conductivity value and process it according to the current invention as detailed herein. The data storage computational algorithm, and the data display can be implemented to the pump operating system or to a remote computing device, as part of the pump's user-interface, mobile application or a separate portal.

The remote computing device 120 may comprise software configured to manage the data received from the monitoring compartment 110. In particular, the software can provide instructions to generate alerts and notifications to a user regarding its breastfeeding care, or in particular, its breastfeeding efficiency, milk production progress and breast health.

In one embodiment of the invention, the software receives conductivity data from the milk sensing device 110, stores the data, analyses the data, and/or displays the data.

As the milk secretory activation biological process is a highly dynamic process, the software can compute parameters based on the conductivity measurements received from the monitoring compartment 110, allowing and reflecting breastfeeding status and progress along the full secretory activation process. For example, in one embodiment of the invention, the milk maturation percentage is calculated and is computed, stored, analyzed, and/or displayed by the herein software.

The software can separately store, analyze and display recorded or computed conductivity data of milk from each breast, distinguishing data collected from milk expressed from either the right breast or the left breast in the scan, allowing monitoring trends in each breast independently, and performing comparison analysis between left and right breast performance, generating side specific alerts and alerting between breasts differences, side preference, or imbalanced milk production.

The software can further analyze the conductivity data or the computational parameters relative to the reference population set, to identify trends within the conductivity data that can be used to define the secretory activation level of the lactating person. In particular, the software can store a pre-defined collected reference population dataset, and computationally compare the measured conductivity level of a tested milk sample to the relevant reference population dataset and generate an output based on the comparison result. In a non-limiting example, the remote computing device 120 can identify that the measured conductivity level of a tested human milk sample is out of a range of the reference population dataset and display an alert accordingly. In a non-limiting example, the alert can be a color indication, e.g., an orange color for delayed breastfeeding status (i.e., MM %, and/or breastfeeding progress is out of range of the reference dataset for the specific day, indicating the delay in milk secretory activation) and green for advanced breastfeeding status (i.e., the mother is more progressed relative to the reference dataset range) or a text notification, and/or a vocal indication. The reference population dataset can also be displayed in the graphs for comparison by the user.

The software can also further analyze the received measured data to generate additional breastfeeding status insights or technical alerts. For example, the received conductivity data can be compared to previous milk conductivity reading(s), can be used to calculate milk progress rate over time, can be used to detect delayed rates when compared to rate reference dataset, and/or can be used to set specific daily goals.

The software can also include the identification of abnormal trends, that the software can mark with suspected technical error tagging or with alerts. In a non-limiting example when the conductivity is out of the range depicted for human milk samples, or when the difference from previous milk sample measurements is not within the rate threshold reference dataset (FIG. 4 E2, the software can push a technical alert relating to an identified issue or condition.

Additionally, in one embodiment, the remote computing device 120 can also provide an interface for logging additional health information, which can be used by the software for generating additional visual outputs, such as parameters and predictions and visual indications. For example, breast pain scale logging is used by a software algorithm for generating a notification regarding breast inflammation in the case of software detected breast differences in the conductivity data and reported pain. In an additional example, the daily breastfeeding sessions can be reported by the mother, and the software can generate specific notifications in case of delayed breastfeeding status, which is identified based on the milk conductivity data together with insufficient reported breastfeeding routine. The software can store specific thresholds for various health related indicators, that can be age-matched for baby age and for enhanced visual output generation.

One will understand that identifying trends within the data can help to correct breastfeeding behavior to improve breastfeeding status.

The stored reference population dataset, and personal data history can be stored in the remote computing device 120, or other web-based storage cache or database on the server.

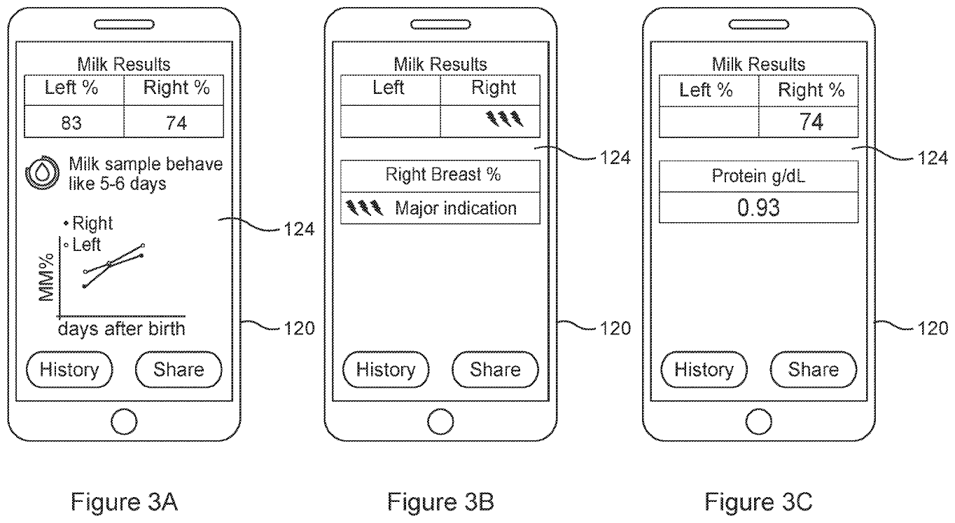

FIGS. 3A-3C depict an exemplary remote computing device 120, such as a smart phone 120 comprising a display interface 124 that is associated with the computer algorithm of the present invention. Specifically, milk related data can be communicated with the smart phone 120 through an internet web page or through a mobile application dedicated to communication with the present invention. FIG. 3A illustrates a display interface 124 with output showing the calculated MM % of each of the right and left breasts. Optionally, display 124 includes a graph that illustrates breastfeeding progress over time with measurements of either or both the MM % and the raw conductivity. Further, a predicted milk sample age or days range after delivery is provided indicating the estimated age after birth calculated according to the measured parameters. FIG. 3B illustrates a display interface 124 with an output alert indicating that there is a health breast issue, such as inflammation on the right breast. FIG. 3C illustrates a display interface 124 with an output illustrating the calculated MM % of the right breast only. Further, a milk estimated concentration is displayed for the tested right breast. It is to be understood that although it is recommended to test both breasts, valid measurements are applicable also for measurements of only one breast. The data can be received either through a connection to the internet or through wireless connection or direct connection with the milk sensing system 10.

The webpage or dedicated software can display, record, and save the milk related data, such as the measured conductivity value transmitted from the milk sensing system 10. The software can also compute parameters based on the recorded value and display them. In a non-limiting example, the software can calculate and display a milk maturation percent parameter. In another non-limiting example, the software can detect/identify and display a breast health issue (e.g., a breast inflammation) alert, as shown in FIG. 3B. In another non-limiting example, the software can compute and display a milk protein level estimation parameter as shown in FIG. 3C.

The display interface 124 of the remote computing device 120 can display conductivity values or calculated parameters relating to the milk measurement for each breast separately, allowing the monitoring of the progress of each breast side independently. For example, FIG. 3A presents a parameter of milk maturation percentage recorded and calculated from milk samples collected independently from the right and the left breasts. The displayed results can be assigned with a color indication that is indicative of sample status relative to the reference population dataset of typical breastfeeding mothers.

The display interface 124 can present a combined milk score calculated based on the measured milk related parameter(s) from both the left and right breasts. As used herein “combined” or “integrated” result is calculated as the best measurement of the left and right breast measurements. The combined milk score is used to calculate the milk sample age after birth (See FIG. 3A). In an embodiment of the invention, displayed data can be presented as a score icon, a colored text or graph and/or a text, and/or via an audio notification. In a non-limiting example, an estimated milk sample age can be calculated by the software and displayed to a user. The estimated milk sample age may be calculated by matching measured milk related values recorded at a specific date to a pre-defined reference population dataset. The user's estimated milk age may be presented as a text 122 depicting the day of the reference dataset that matched to a recorded milk measured value. A color indicative of the matched milk after birth age and the actual baby's age is shown in FIG. 3A.

In some embodiments, the display interface 124 can present a graph monitoring the conductivity or MM % data of the milk over time, for each breast separately. The graph can optionally include benchmark reference dataset for comparison, as depicted in a typical breastfeeding mother and shown in FIG. 3A.

In some embodiments, the display interface 124 can indicate a breast health issue such as breast inflammation as shown in FIG. 3B. In some embodiments, an estimated protein level can be presented by the display interface 124, as shown in FIG. 3C.

Additionally, the remote computing device 120 can display historical information relating to the received data. For example, the smart phone 120 can display a log of all the scans by date, days from birth, results and color indications. This feature can allow the mother or caregiver to see the values in real-time and to be proactive in the mother's approach to breastfeeding care. For example, the saved data can allow the mother or the caregiver to notice early trends of delay in milk maturation relative to baby age, that could prove a useful method for detecting early breastfeeding status related issues, that can lead to complications such as low milk supply and slow weight gain in the baby. These visual outputs can be provided as values, color indications, icons, text, and/or audio notifications. One will appreciate that there are various methods for smart phones to alert a user, all of which can be used within the present invention. These values can be visualized alongside the reference population dataset to allow the user to compare personal results to the reference population dataset and identify trends. These trends can also be identified over a progress or rate in the form of graphs or history charts that display historical milk data.

Visual outputs of system 10 can also include detecting breast health issues such as breast inflammation in a specific breast, according to changes in values between daily measurements, or in a case where there is breast pain. In a non-limiting example, the display interface 124 can present a text alert for a detectable sudden one breast deviation and breast inflammation trends, and provide instructive guidance for improving general breast health, such as, a breast massage and/or breast emptying and/or a need for other assessment.

Additionally, in a non-limiting example, the herein visual outputs provided by system 10 can also include estimations regarding one or more nutritional composition of the milk sample, such as protein level estimation. In some embodiments, protein levels in a milk sample are determined based on empirical correlation between milk maturation and/or milk conductivity and protein levels in milk samples following birth. In a non-limiting example, the display interface 124 can provide an output indicating a protein level estimation for aiding the assessment of required individual targeted fortification of human milk.

The algorithm of the system 10 is also configured to select the visual outputs presented to the user=by computational conditions relating to other breastfeeding and infant data recorded by the mothers, generating augmented personal visual outputs in the form of a text, progress records and charts. These can aid the mother or the caregiver in breastfeeding management and proactivity.

To allow for a remote guidance and support, the software of system 10 can comprise a sharing feature that enables a user to easily share the recorded milk related parameters and all other user collected data and analysis, including breast health issues and breastfeeding status with another entity, such as a caregiver or a lactation consultant, or a physician. A sharable health report can include data, but is not limited to, the milk related data such as the conductivity values or the milk maturation records and outputs including baby's weight gain records, and the maternal recorded symptoms and breastfeeding habits. Technical error alerts generated by the monitoring compartment 110 can additionally or alternatively be triggered by the remote computing device 120 and presented via display interface 124. In a non-limiting example, technical error alerts triggered by the processing unit 250 of the monitoring compartment 110, such as low milk sample volume, trigger error messages on the smartphone, prevent result presentation, and alert or notify the user that another milk measurement should be performed. Additional technical error alerts beyond the errors received from monitoring compartment 110 are also contemplated herein. These include measurements from a breast that are too low or high, i.e., beyond or above a limit threshold, compared to a previous measurement. The system 10 then assumes that something went wrong during the measurement, although no other technical alerts were detected by the monitoring compartment 110, the user then gets a technical notification advising her to make a new measurement before saving.

FIG. 4 illustrates a flow chart depicting various scenarios of breastfeeding monitoring utilizing the herein system 10. As used herein the term “reading” can be used interchangeably with the term “measurement”. System 10 provides an algorithm providing various visual outputs, alerts, and insights according to the measured milk sample conductivity.

System 10 evaluates if the milk conductivity measured is valid by several steps of detecting technical errors that can present the relevant alert to the user. If no technical errors are detected the measurement is considered Valid. Valid conductivity measurements can be processed by calculation to provide a milk maturation score, wherein the milk maturation score can either fail or pass a defined predetermined threshold and can be logically assigned as an ‘advance’ or ‘delay’ visual output indicative of breastfeeding status. In addition, a conductivity measurement can be logically compared to one or more stored readings from the other breast, and logically assign a status or display a visual output indicating that there is a difference between the breasts or that a breast health issue such as inflammation has been detected if a value passes a specific predetermined threshold. Moreover, a conductivity reading can be compared to a previous stored reading(s) from the same breast and assigned a status of ‘low’ or ‘high’ rate of progress, based on whether it passes or does not reach a specific predetermined threshold.

Returning to FIG. 4. the system 10 detects a reading/measurement (a) that can be assigned as a reading/measurement that is out of a certain threshold range (b2) which leads to a technical alert (oval black box). A reading can be within a certain threshold range (b1) leading to a valid measurement that generates a visual output regarding a milk related parameter to display (c1). A valid conductivity measurement triggers visual outputs of the calculated milk maturation (g1), and/or the calculated protein level (g2).

In one embodiment, ‘delay’ or ‘advance’ breastfeeding status values or visual outputs are generated by the herein algorithm by comparing a reading to daily reference thresholds, where ‘delay’ (d2) is when a reading failed a daily reference threshold and ‘advance’ (d1) is when a signal is at or exceeds a daily reference threshold. As used herein the term “daily reference(s)” refer to conductivity or maturation reference datasets derived from a database of empirically collecting and testing human milk samples from breastfeeding mothers at different days after delivery. By “delay” or “advance” it is meant to provide an indication to the woman regarding her breastfeeding status progress.

As described supra, reading records from one or both breasts can be stored in system 10. The algorithm may compare current readings to previous readings. The algorithm may compare a reading from one breast to one or more previous reading(s) of the same breast. In one or more embodiments, readings between breasts (right and left) can be compared. If the difference between the breasts exceeds a certain threshold value (f1), the system assigns a visual output indicating that there is a side difference. Further, if there is a record of pain or any other health related issues in the system an inflammation visual output may be generated.

In one embodiment, when previous reading records from milk taken from the same breast are stored, the system assigns visual status values of low or high breastfeeding progress rate by logically comparing the current reading to the previously stored reading(s) from milk taken from the same breast. This comparison determines if the difference is out of a specific daily reference rate threshold (f2). In one embodiment, the daily references used by the system are conductivity rate datasets generated empirically by collecting and testing human milk samples from typical breastfeeding mothers and establishing conductivity rate references for different days post-delivery. If a reading is above the threshold reference, a “high-rate” visual output is assigned. If a reading is below the threshold reference, the reading has failed the threshold test, and a “low rate” visual output is assigned. The low and high-rate outputs provide insights to a user on whether or not she is progressing according to expected in her stage in accordance with dataset derived from healthy and efficiently breastfeeding women.

One will understand that the described logics include, without limitation, the raw measurement, the adjusted conductivity parameter, and/or a calculated parameter based on the conductivity, such as the milk maturation percentage. It is to be understood that the herein thresholds used by the herein software correspond to the same type of parameter being compared. By “passing” a threshold it is meant to refer to a higher or lower than the indicated reference threshold, depending on the nature of the compared parameter.

Abnormal conductivity readings that trigger a technical alert (b2) can be either conductivity values, which are above, or below realistic values set, defined for the full physiological range of human milk conductivity, or defined as daily references set for the specific day following delivery. In addition, readings can be assigned by the logic as a technical alert of ‘Not enough sample’ (c2) if a second parameter of ‘Sample level detection too low’ is assigned to the reading. In one embodiment of the current invention, the absence of a signal in the second electrical circuit that indicates of insufficient milk in the sample holding chamber can generate by software an error output of ‘Sample level detection too low’ that is assigned to the reading.

One will understand that these are just potential reasons that an abnormal reading can occur and are not meant as an exhaustive list of the abnormalities that the present invention can identify and compensate for.

Returning to FIG. 4. in block “a” a reading is detected. In one embodiment, upon detecting a conductivity reading, the software can determine if a reading is within a valid reference threshold range. If the reading is within the reference threshold (block b1), the software can determine if enough milk sample volume was detected by the monitoring compartment 110. For example, the software, utilizing processing unit 250 of monitoring compartment 110 can detect if the sample volume is sufficient for a valid reading when a signal is absent. If sample level detection is too low, an error is assigned to the reading, and the read result is not further analyzed or displayed by software as it may represent an inaccurate reading (c2).

In response to a reading out of the range of the full reference threshold (block b2) or when the software detects that the milk volume was not enough (reading assigned an error of ‘Sample level detection too low’, block c-2), the software can generate a visual indication of a technical alert, and send an indication on the remote computing device 120 or on the monitoring compartment 110. This alarm can be a different alarm than the alarm used for a verified reading. For example, the software can generate an alert message on a remote computing device 120, or the software can display a visual indication on the monitoring compartment 110 such as an LED alarm. Technical alerts are used by the software to notify the user that a reading is invalid, and that a rescan is needed.

Returning to block b1 in FIG. 4, when a reading is valid and a sample level is detected (block c1) and software has determined that the signal from the conductivity sensor is valid (i.e., the milk sample volume in the cell was sufficient and conductivity was validly measured), a reading can be presented (block g1), and can enter the next logic decision in the software that determines the breastfeeding status of the reading, whether it is defined as ‘delay’ (d2) or ‘advance’ (d1) relative to the daily references.

‘Delay’ or ‘advanced’ breastfeeding status values and/or visual outputs are generated by the software by comparing readings/measurements to a stored reference dataset corresponding to each specific day after delivery, or to a range of days after delivery wherein delay is when a reading failed or is below the threshold of the daily references dataset (block d2), and an advanced visual output is assigned when a reading is at about or is above the daily reference values (block d1). In one embodiment, as used herein “daily reference datasets” are milk conductivity benchmarks generated empirically by collecting and testing human milk samples from typical breastfeeding mothers at different days after delivery. Visual output can be displayed as a numeral figure, a color alert, an audio alert, or a text message display on the remote computing device 120, or within a display of the monitoring compartment 110 if applicable (not shown in the Figures). In one embodiment, the ‘delay’ outcome is the highest priority and may be assigned an alert by the software. Specifically, an alarm may include a LED light and may comprise a distinct color.

In one embodiment, protein level estimation is calculated, and visual output is presented as shown in Block g2 in FIG. 4. In one embodiment, protein estimation levels are generated by a mathematical calculation pre-determined empirically by correlating conductivity and protein levels in a dataset from early postpartum human milk samples.

Similar to the above, the software can determine additional technical alert notifications in case of a reading over the extremes of the daily thresholds (block e1) or in a case where a difference from Recent Sequential reading passes the threshold (block e1). In one embodiment, this can generate a message notification display on the remote computing device 120 with the displayed outcome (block d1 or block d2), to notify the user of a possible questionable reading.

In the case where there are previous readings from the same user stored, software can further generate additional visual outputs.

When scanning milk from one breast, if reading records from the other breast are stored, the software compares the reading to the record of the last reading of the other breast, and if the difference passes a specific threshold stored in the software (block f1), the software assigns a ‘side difference’ outcome. If a breast pain indication is recorded with the reading, and the software assigned by the logic ‘a difference from the second breast above threshold’ (block f1), the software can generate an ‘inflammation-note’ output. In one embodiment, the ‘side difference’ status and/or the ‘inflammation note’ status can generate a message notification display on the remote computing device 120 with the displayed outcome, to notify the user of a possible breast related health issue.

When readings of previous records associated with the same breast are stored, the software can calculate the conductivity change rate/progress or maturation % change rate/progress, defining the conductivity change from the last previous date measurement over time elapsed, and compare the computed conductivity change to a specific reference rate threshold stored in the software (block f2). When by logic a reading passes a rate threshold, a high-rate outcome is assigned, while when a reading fails a rate threshold, a low-rate outcome is assigned. In one embodiment, the high and low-rate outputs can generate a message notification display on the remote computing device 120 with the displayed outcomes, to notify the user of a possible trend in the breastfeeding progress. In one embodiment, the software can also set personal targets based on the rate output, to set reachable goals and intermediate milestones for tracking milk maturation progress.

In one embodiment, the breastfeeding monitoring system 10 can provide the milk monitoring compartment 110, in communication with a variety of other breastfeeding aids, specifically aids designed to measure milk volume transfer in a single breastfeed, for example milk flow meters, breast pumps and pumped milk meters, and baby scales. In one embodiment of the present invention, a breastfeeding monitoring system 10 can include one or more of these devices in combination. For example, the milk monitoring compartment 110 can detect a ‘delay’ breastfeeding status and can combine a notification with a milk volume transfer in a single feed or with a baby weight gain outcome, to help the mother and a caregiver to better determine the notification and the severity of the delay, and better plan breastfeeding care intervention.

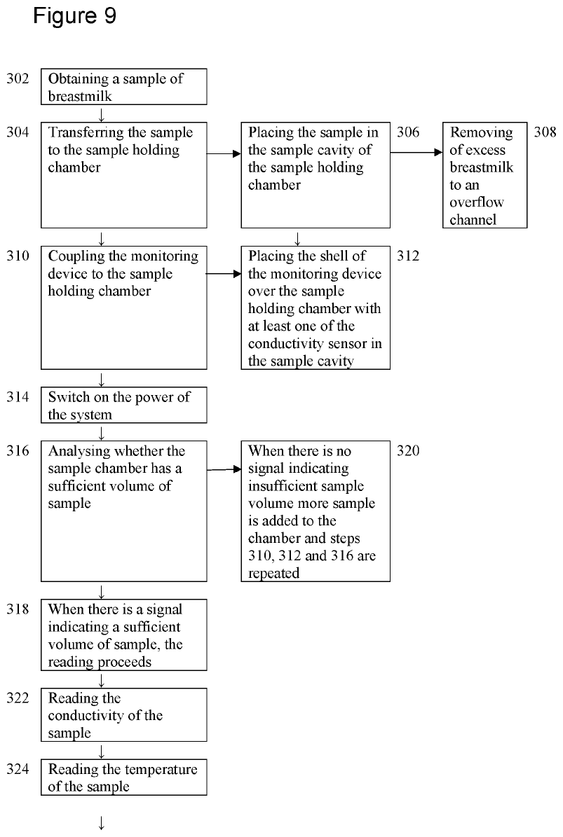

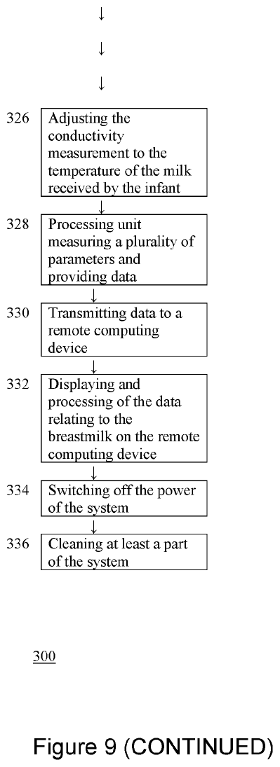

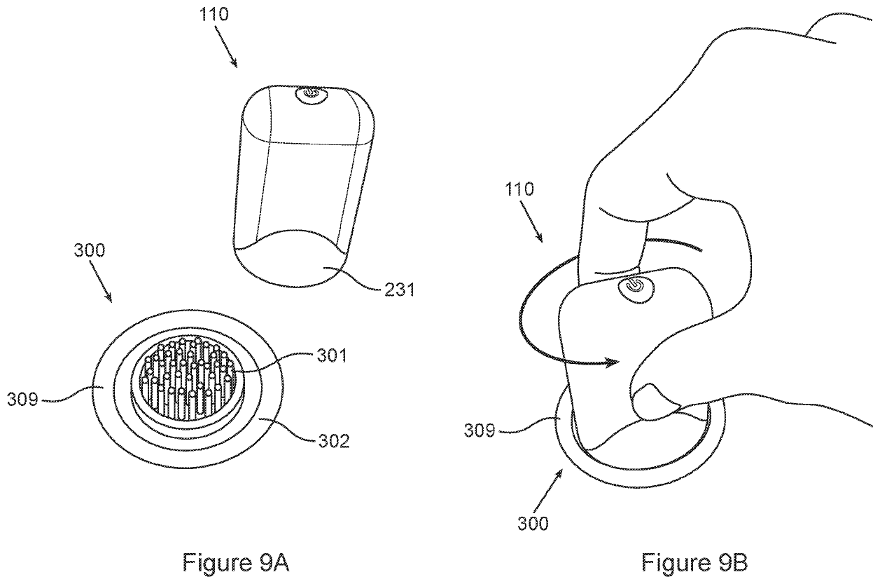

The present invention provides a method 300 of using a breastfeeding monitoring system 10 of the present invention as shown in the flow chart of FIG. 9. The order of the steps is not meant to be limiting and any suitable order may be used. A sample of breastmilk is obtained 302, The breastmilk may be a sample of the breast milk expressed from a lactating individual. The small sample volume required enables the use of the system as early as the first day postpartum. The sample may be obtained by pumping or expressing the milk from the breast by any effective method including manually or with a breast pump. In some embodiments a breast pump is incorporated with the system of the present invention. The sample may be contained in a receiving container and may be transferred to a sample holding chamber of the present invention 304. In some embodiments the sample holding chamber and a monitoring compartment may be included in a designated system container for storing the system and the components may be removed from the container before use. A volume of the sample may be poured or pipetted into the sample cavity of the sample holding chamber 306. Care is taken to add a volume which is in the required range for effective reading by the system of the milk sample. In some embodiments a volume of from about 300 microliters to about 350 microliters is added. Any excess milk is removed by the overflow mechanism 308, which may be configured to include outlets on the wall of the sample cavity positioned above the maximal sample level of the sample cavity and whereby the outlets are connected to an overflow channel for storing the excess sample. The monitoring compartment is then coupled to the sample holding chamber 310. In some embodiments this is done by a user placing the shell of the monitoring compartment over the sample holding chamber such that the base of the shell is held by a corresponding walled cavity at the base of the sample holding chamber and such that at least one conductivity sensor of a sensing module is immersed in the sample cavity and in the milk sample 312. The force of the sensing module on the sample may displace a small amount of the sample pushed out by the force over the sample maximal level to the overflow mechanism ensuring a fixed volume for performing a reliable measurement. The system is now ready for reading of the sample. The system is switched on 314 to supply power to the system. The system analyses whether there is a sufficient volume of milk sample to provide a reliable reading 316. This is done by a suitable method, such as described in FIG. 8A and FIG. 8b whereby a sufficient sample level is achieved, when the sample touches the conductive surface of a temperature sensor metal case in the sensing module and completes an electrical circuit with one of the conductivity electrodes. The electrical circuit can provide a signal to the system, which is interpreted as the reading can proceed 318. If the sample is insufficient a user can add more sample and repeat the described steps 320. When the sample is sufficient a conductivity measurement of the milk sample is read 322. The temperature of the sample is measured 324. The conductivity of breastmilk may change at different temperatures. As such, for accurate analysis of the milk measurements, the conductivity should be corrected to a value that corresponds to a fixed predetermined temperature, which may be for example, ambient temperature to enable reliable comparisons 326. The system can employ the processing unit of the monitoring compartment as previously described to measure a plurality of parameters including breastfeeding status and to identify various breast health conditions and present corresponding visual outputs 328, such as milk conductivity MM %, estimated milk after delivery age, protein level, breastfeeding status in each breast and a comparison therebetween. In some embodiments, the system of the present invention transmits the data to a computing device, which may be a remotely located device such as a smartphone 330. The computing device includes a software program or a computer application of the present invention. The software can provide milk sample related data to a user and may include graphical displays, numerical displays, text displays, and/or audio notifications 332. The software can include a database of previous readings of the user and may have a log of data referring to each breast and the milk sample expressed from it. The software may also include a database of benchmarks and normal and abnormal ranges of values of breast related parameters from data collected from a suitable population of lactating individuals. In the case of the system of the present invention identifying values of parameters, which are not in a normal range, the software may also provide instructions on how to improve the value of the parameter for more efficient breastfeeding. In some embodiments, the data can be used by a caregiver to overcome identified problems, to identify additional factors and improve at least one of the milk volume, the milk quality, the health of the lactating breast and the efficiency of infant latching, breastfeeding frequency, sleep schedule and medical interventions. The data may also find use in research relating to breastmilk, breastfeeding and breast health. The system of the present invention is configured to be used immediately at home, or anywhere indoors or outdoors, e.g., at a point of care. The very small volume that is needed by the system enables its use even very soon after the birth of the infant. The method of using the system and the analysis of the milk is very quick and can be done instantly, for example in less than a few minutes, for example, in less than about 5 minutes, or less than 4 minutes, or less than 3, or less than 2 minutes, or less than a minute. This promotes use of the system. By routine and daily monitoring of the milk with the system, breastfeeding related problems can be detected very early, which aids in an early solution and improves the chances of avoiding adverse effects on the baby's health or the lactating individual's health. The early and regular monitoring also helps a lactating mother avoid or overcome problems, which may have made her stop breastfeeding or made her breastfeeding experience a negative one. After use of the system, the system can be switched off 334 and the monitoring compartment and the sample holding chamber can be separated. In some embodiments the sample holding chambers are for one time use and can be freely disposed. Components of the device may be cleaned 336. In an embodiment wherein the sample holding chamber is for repeated use, the holding chamber may be suitably cleaned. The monitoring compartment may be cleaned. In some embodiments only the sensing module of the monitoring compartment is cleaned.no. In some embodiments, the present invention provides a cleaning device which is suitable for cleaning parts of the monitoring compartment, which are configured for contact with the sample.

Accordingly, FIGS. 1-4 provide a number of components, schematics, and mechanisms for monitoring breastfeeding efficiency of a lactating woman. In one embodiment, a wireless sensor communicates data from a milk sample to a remote computing device 120. The remote computing device 120 can then be used to monitor the breastfeeding efficiency, set milk secretory status, or can alert an individual to a negative trend and progress over time, by analyzing trends in the received milk data. In one embodiment, alarms can be detected and alert a user to breast health related conditions, such as inflammation. In one embodiment, technical alarms can be detected and alert questionable readings for reducing false alarms and increasing the system implementation probability.

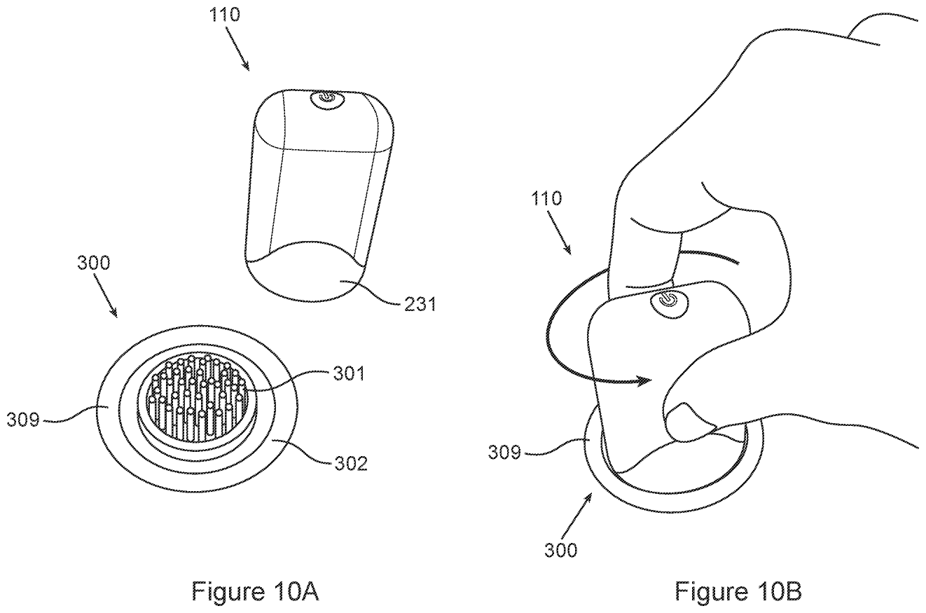

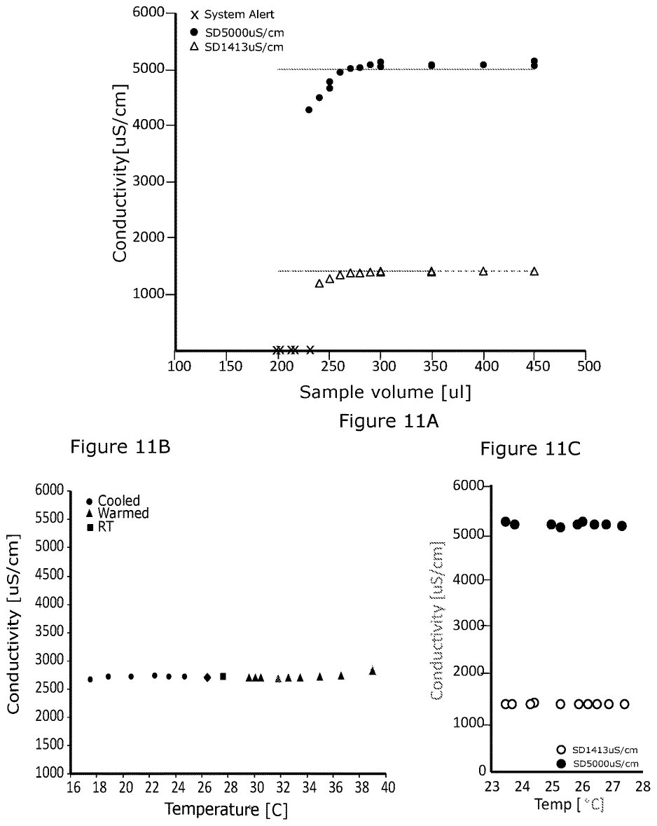

FIGS. 10A-10B illustrate a cleaning tool 300 with one or more vertically positioned cleaning teeth 301. The cleaning teeth 301 may be centered in the middle of a cleaning tool base 311 to allow for an efficient cleaning of the sensing module 208. There may be a walled ridge 309 on the base 311 of the cleaning tool for correct coupling and placement of the milk monitoring compartment 110 in relation to the cleaning tool 300. The cleaning tool 300 is preferably used following each milk sample measurement.

Reference is made to the following examples, which together with the above description illustrates the invention in a non-limiting fashion.

### EXAMPLES

### Example 1—System Use Case Example

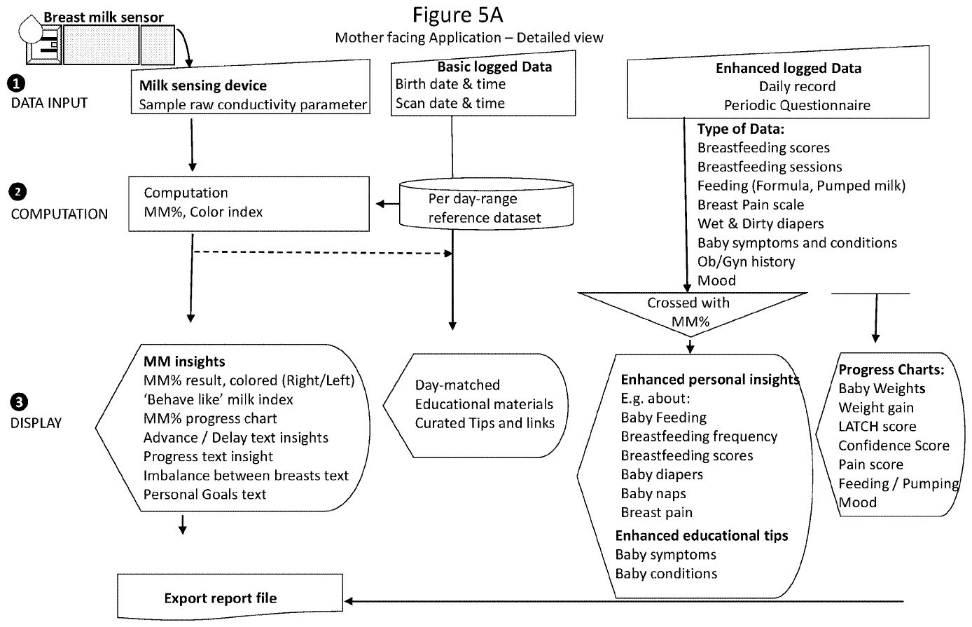

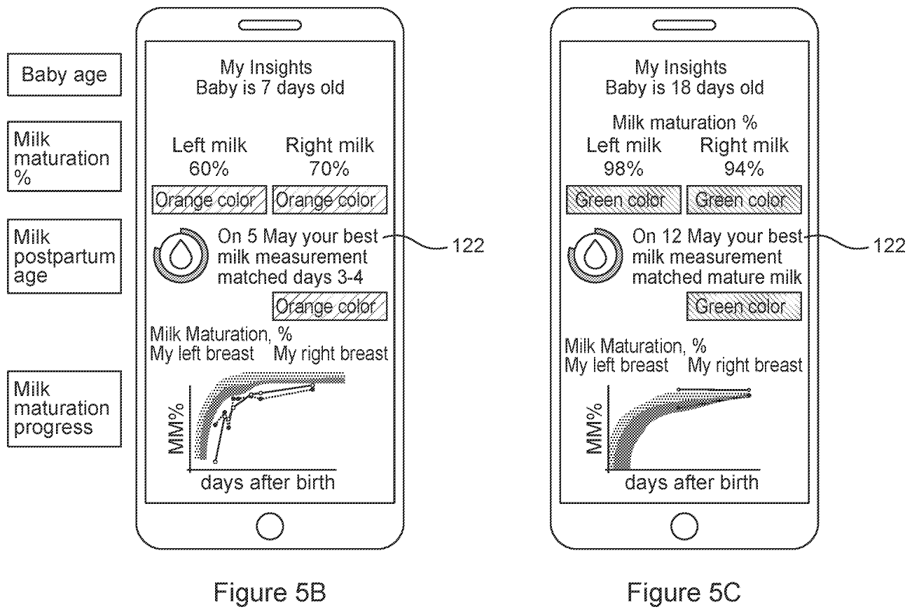

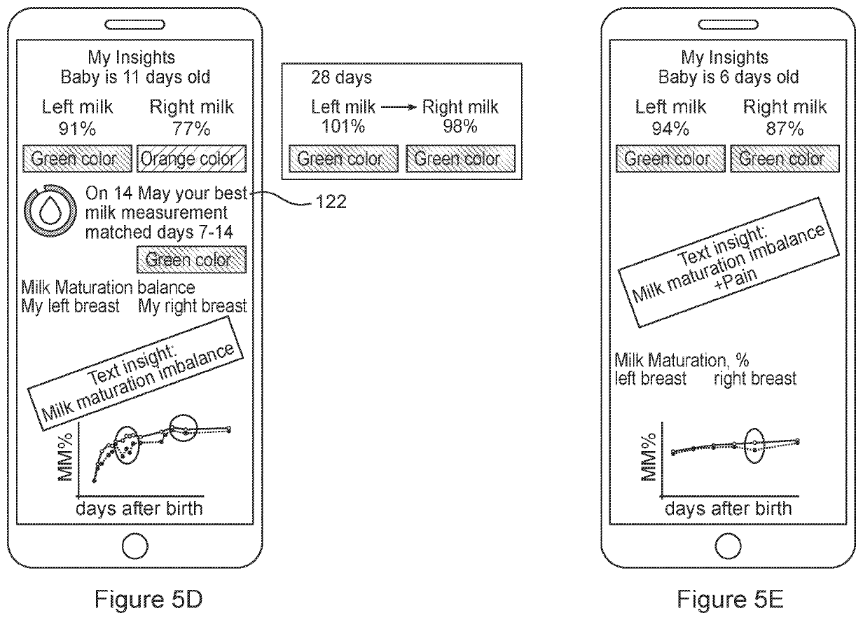

In the following non-limiting example, the system of the present invention was used in a group of real-world breastfeeding mothers, each mother was provided with a milk sensing system of the present invention and asked to download a dedicated smart phone application, of the herein software. Upon using the system of the present invention, the mothers' user interface would instantly compute and display the calculated Milk Maturation Percentage (MM %) for any logged milk scan, while also allowing for additional data entry. FIG. 5A shows the exemplary mother app interface flow chart.

The app's dashboard showcased the most recent scan from each breast separately, featuring a color indication and a “behaves like” parameter for easy interpretation, in comparison to a reference population dataset representing successful breastfeeding. For each case, multiple snapshots of the mother-facing app are presented. Examples of mothers where the system's usage and MM % parameter effectively identified, monitored, and supported the breastfeeding journey (FIG. 5B-E).

Instances demonstrating milk maturation delay were observed through app outputs (numbers, colors, text), which correlated with reported breastfeeding challenges, such as inadequate milk supply and slow infant weight gain (FIG. 5B). Cases showcasing milk maturation adequacy were also evident in app outputs, aligning with satisfactory breastfeeding experiences and appropriate infant weight gain (FIG. 5C).

Additionally, examples where the system detected differences between the mother's left and right breasts, consistent with the mother's self-reported observations (FIG. 5D-E) are presented. In some cases, these discrepancies were associated with milk supply differences, further resolved by active pumping (FIG. 5D), while in others, they were associated with reported breastfeeding pain suggestive of inflammation and generated a relevant alert (FIG. 5E).

### Example 2—Feasibility of Using Real World Data Collected with the System of the Current Invention Used in a Home Setting for Gathering and Analyzing Valuable Secretory Activation Data

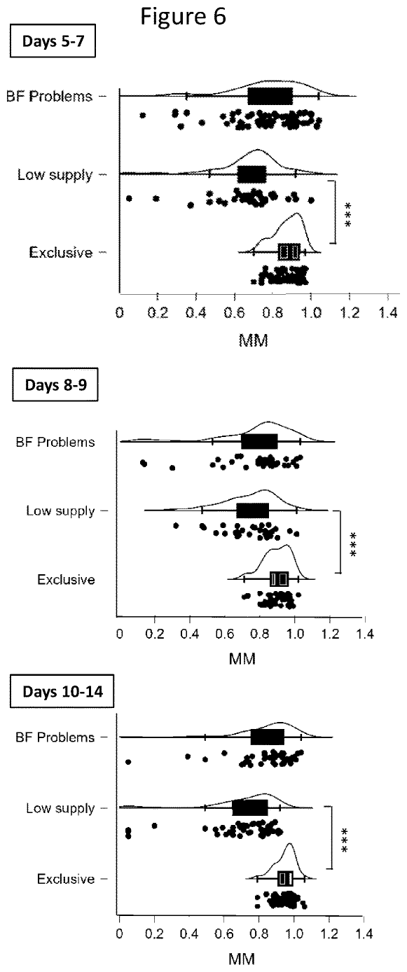

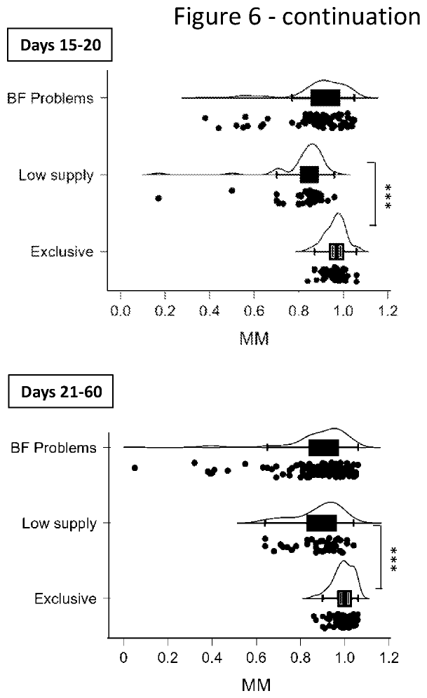

An additional non limiting example demonstrates the feasibility of using real world data collected with a non-limiting version of the current invention used in a home setting for gathering and analyzing valuable secretory activation data (FIG. 6). Analysis of the data from home use of the device of the present invention was able to demonstrate lower milk maturation % values associated with early breastfeeding problems indicative of low milk supply. A dataset was retrospectively classified by authors to one of three breastfeeding classes based on user records of breastfeeding exclusivity and reported breastfeeding problems, namely, ‘normal’; ‘breastfeeding problems’, and ‘low supply’, representing milk supply problems ‘severity’. A retrospective analysis in which the ‘normal’ dataset as described above was compared to the dataset of records classified as ‘low supply’ (253 scan records). The low supply MM % array revealed a tendency to lower values compared to the ‘normal’ Predominant Exclusive breastfeeding classification with a lower group median, quartiles and mean MM % for ‘low supply’ vs ‘Normal’ groups throughout the full period. Per day-range analysis, revealed lower mean MM % in the ‘Low supply’ classified records vs the ‘Normal’ classified group in every day-range analyzed (Two factor ANOVA P<0.001, Breastfeeding classification P (Tukey)<0.001). A post Hoc analysis showed a strong statistical significance between datasets at day 5 onwards after birth (Day 5-20 P<0.001; Day 20-60 P=0.002).

### Example 3—Demonstrating the Feasibility of Using Milk Sample Conductivity Data Collected with a System of the Current Invention, for Estimating Human Milk Sample Protein Levels Compared to a Laboratory Grade Milk Protein Assessment Method

An additional non limiting example demonstrated the feasibility of using milk sample conductivity data collected for estimating human milk sample protein levels compared to a laboratory grade milk protein assessment method. FIG. 7 presents the correlation between conductivity values as measured by the current invention and protein levels as directly measured by laboratory methods offering the ability to estimate protein levels based on milk conductivity, implemented in the current example. (FIG. 7).

TABLE 1
a table is shown depicting the predicted/projected protein
content as calculated by the herein invention compared
to actual (direct) protein assessments, in accordance
with an aspect of the present invention. The average protein
recovery achieved was ~106% (range of 88-115%).

| | | Lab grade | Projected | |
|---|---|---|---|---|
| Sample | Milk Cond | protein | protein | % |
| ID | sensor | [g/dL] | [g/dL] | Recovery |
| 14060 | 1596 | 0.62 | 0.71 | 115 |
| 14015 | 1639 | 0.70 | 0.73 | 105 |
| 13029 | 2900 | 1.28 | 1.36 | 106 |
| 14127 | 1789 | 0.70 | 0.81 | 115 |
| 14017 | 2644 | 1.40 | 1.23 | 88 |

### Example 4—Demonstrating the Feasibility of Using Milk Sample Conductivity Data Collected with a System of the Current Invention, for Measuring Conductivity in Tiny Volumes of Human Milk Sample

As colostrum and early milk are produced in small volumes during the first days after birth, the herein device was engineered to reliably sense conductivity in several drops of human milk. The herein system and device was demonstrated to have excellent reproducibility (CV95%<5%) and accuracy (error<5%) for conductivity measurements of a small specimen (350 μl), with good device stability and almost perfect inter-device unit reliability (ICC>0.90). With regression analysis, an excellent agreement between milk maturation (MM %) output or its raw conductivity signal and laboratory measurements of conductivity and sodium [Na+] in a dataset of milk specimens (n=167; R2>0.9) was illustrated. A 350 μl specimen size was used for consistent laboratory testing.

To demonstrate measurement reliability in a range of milk volumes tolerated by the milk holding chamber, conductivity was assessed for a range of specimen volumes, and found consistent above 260 μl. Error introduced with specimen volume under 230 μl system generated insufficient specimen alert in line with system design (FIG. 10A).

The system was engineered to measure local temperature and to automatically correct the measured conductivity to 25° C. To test the reliability of the results at a range of temperatures, the autocorrected conductivity measurements were evaluated either by monitoring the changes in specimen temperature after warming or cooling or by comparing various samples at a range of ambient temperatures; consistent results were found between 17° C. and 39° C. (FIG. 10B-C).

### Example 5—System Performance Testing

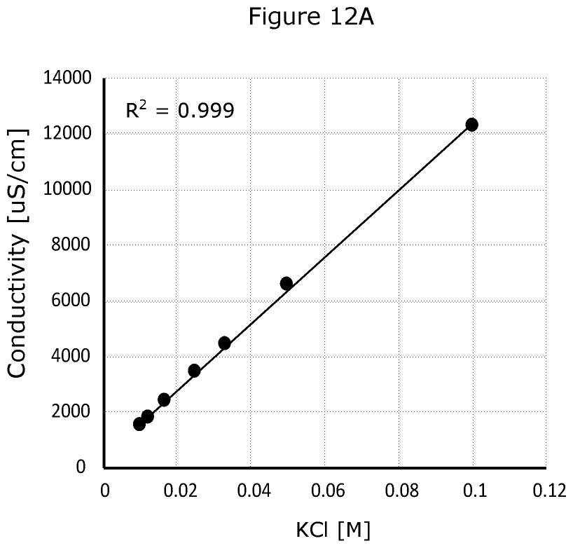

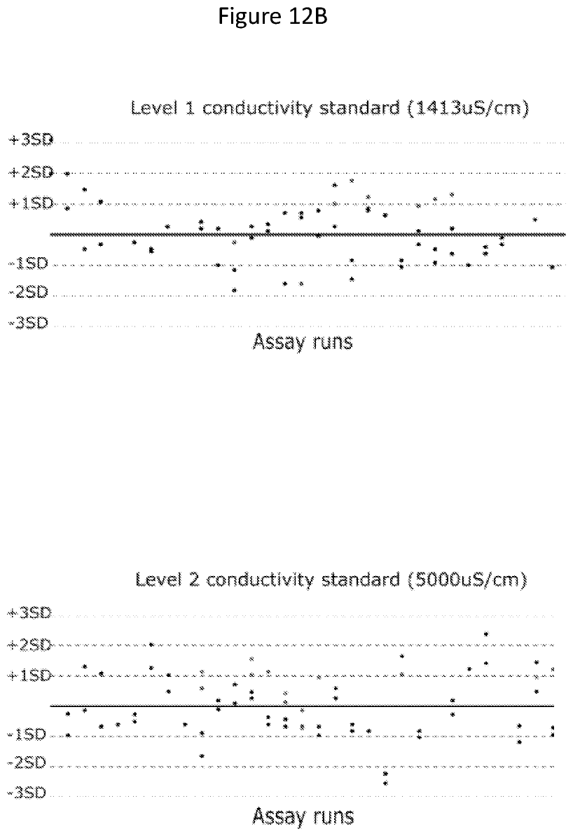

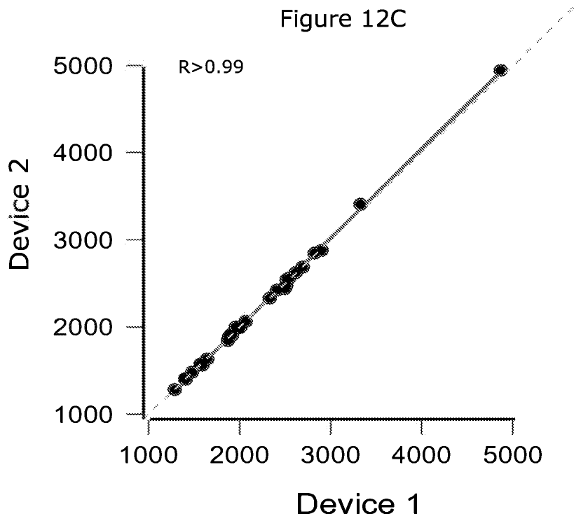

The system was engineered to cover the full physiological range of breastmilk conductivity measurements. Conductivity linearity was confirmed in the measuring range of 10-100 mM KCl corresponding to the physiological range expected in breastmilk throughout the breastfeeding stages (FIG. 12A; R2=0.999). The system is designed to generate an alert when the specimen's conductivity is out of the full expected range of a reference population dataset, as this may imply specimen dilution or external contamination.

System performance was evaluated for precision and accuracy of conductivity measurements using two levels of conductivity standard solutions (Level 1 standard material (1413 μS/cm) and Level 2 standard material (5.00 mS/cm)). The variability and stability of the conductivity results were evaluated over a period of intensive use without re-calibration. The evaluated dataset included 599 measurements generated by routine quality assurance tests of 5 different device units, assessed over a 2-month period, with 32 independent recording dates. A representative Levey-Jennings chart of the quality control data logged by one of the device units demonstrate stable reads over time (FIG. 12B).

System reproducibility was evaluated with data from mother's milk measurements, assessing 24 agreement between different device units in a dataset of a total of 112 mother's milk measurements in an extended physiological range generated by comparing conductivity records of 28 mother's milk specimens by 4 device units. By test-retest analysis, an 2 almost perfect correlation (R>0.99) (FIG. 12C) and excellent agreement (ICC=0.99) between device units was demonstrated.

### Example 6—Validation of the Device with Na+ Ion Selective Electrode Instrument

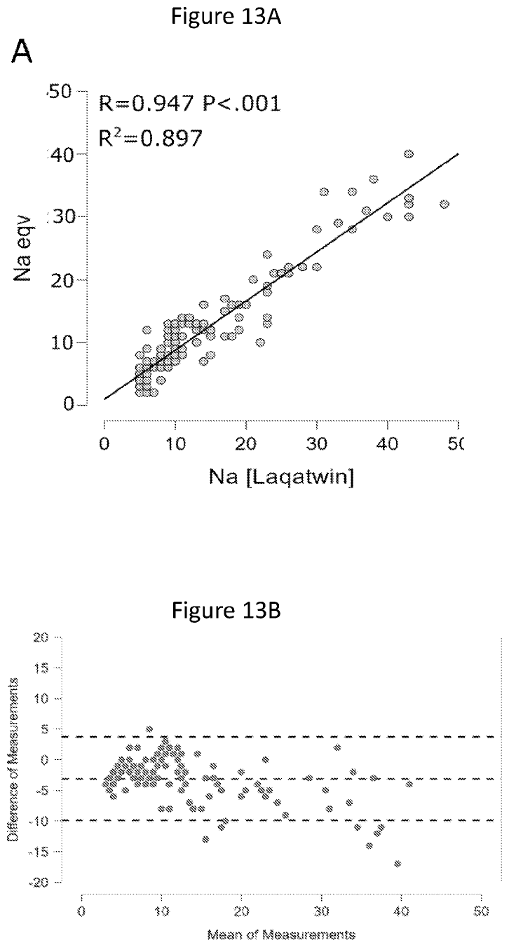

As measuring of Na+ is being increasingly employed in research assessing lactation success, the dataset was further evaluated for the degree of agreement between methods using the Bland-Altman plot, a useful display of the relationship between two paired variables using the same scale. For enabling the analysis, an estimated sodium equivalent (‘Na eqv’) parameter was calculated for a subset of specimens (n=132) according to equations derived from conductivity and Na+ records measured in a separate set of breastmilk specimens by laboratory methods (n=72, 22 [23]). Differences are within the theoretical limits of agreement; the points are centered around a mean difference of −3 mmol/L and provide a reasonable confidence interval (+1.96 SD; −9.9, 24+3.7 mmol/L) (FIGS. 13A-13B).

### Example 7—Exemplary Method for Setting Reference Dataset of Milk Maturation % from a Population of

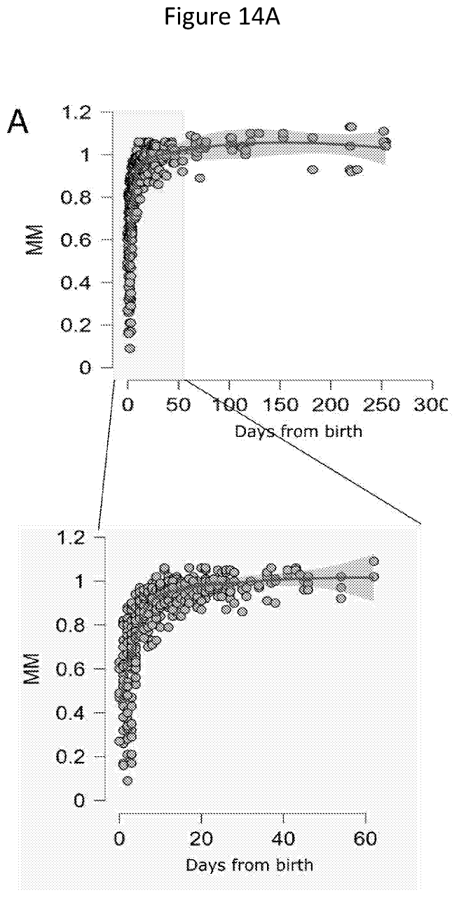

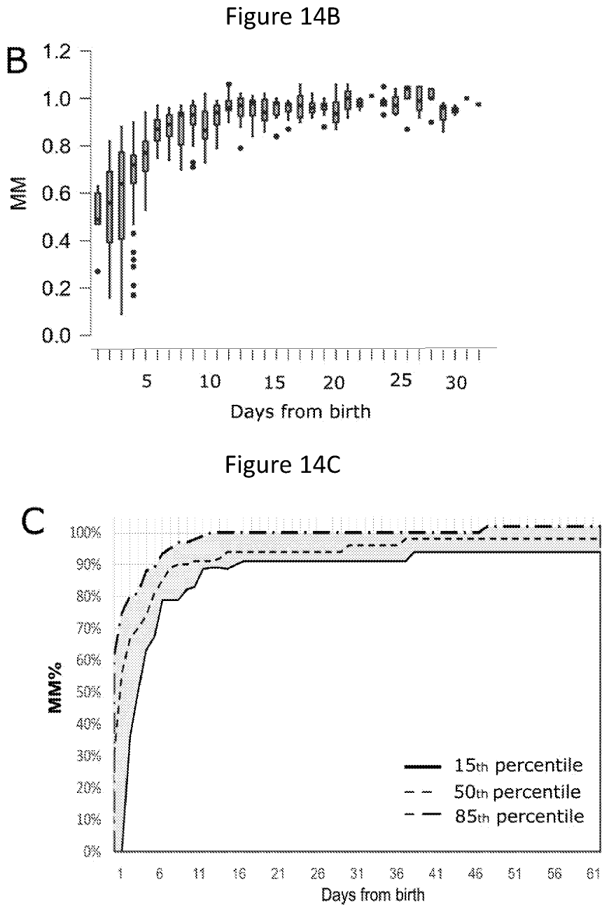

A dataset of 507 scan records tagged as exclusive breastfeeding, full breastfeeding, and predominant mother's own milk (≥80% of daily feeds, also referred to as high partial breastfeeding) at the time of the scan, with no breastfeeding problems (“reference population dataset”) was collected. Data analysis revealed rapid elevation in the MM % parameter in the first days from birth that continued to increase slowly along 2 and 3 weeks postpartum, reaching a stable plateau of approximately 100% (FIG. 14A-C). A set per-day MM % “normal” percentiles were defined at the 15th, 50th, and 85th percentiles for each day/day range (daily in the first 21 days postpartum and at 7-10-day intervals at days 21-60 and >60 postpartum) (FIG. 14C). These calculations and visualizations were further used as references for single or multiple sets of measurements.

The present invention may be implemented using a general-purpose or special-purpose computer, which may include various computer hardware components as discussed below. Additionally, the present invention may be embodied in computer-readable media that contain computer-executable instructions or data structures.

Computer-readable media that can be accessed by a general-purpose or special-purpose computer may include RAM, ROM, EEPROM, magnetic disk storage, optical disk storage, or any other medium capable of carrying or storing a desired program code means in the form of computer-executable instructions or data structures. Moreover, any network or communications connection (hardwired, wireless, or a combination thereof) through which information is transferred to a computer can also be viewed as a computer-readable medium.

Computer-executable instructions may include instructions and data that cause a general-purpose or special-purpose computer or processing device to perform one or more functions. While the subject matter has been described in terms of structural features and methodological steps and acts, it is to be understood that the invention defined in the appended claims is not limited to these specific features or acts.

The present invention may be embodied in other specific forms without departing from its essential characteristics or spirit. The described embodiments are intended to be illustrative, rather than restrictive, and the scope of the invention is defined by the appended claims. All modifications falling within the meaning and range of equivalency of the claims are intended to be embraced within their scope.

1. A system for monitoring breast milk at various stages of breastfeeding from colostrum through transition to fully mature milk in a human subject, the system comprising:
a monitoring compartment for measuring milk related parameters in a sample of milk from the subject, whereas the sample is held in a milk sample holding chamber, the monitoring compartment comprising:
a sensing module comprising a set of at least two electrodes for measuring conductivity, a volume sufficiency sensor, and a temperature sensor, the sensing module configured to provide milk related parameter measurement(s) relating to the conductivity, the temperature and the volume sufficiency of the milk sample;
a processing unit in data communication with the sensing module, the processing module configured for processing the conductivity, temperature and volume sufficiency of the milk sample and for providing a milk status value relating to the breastfeeding efficiency of the subject; and
a shell housing comprising a shell body and an open shell base, the shell housing encapsulating the sensing module and the processing unit within the shell housing cavity, and wherein the sensing module is positioned within the shell housing such that at least the sensing portion thereof extends down vertically from the body of the shell housing towards the open shell base; and
at least one sample holding chamber, the sample holding chamber comprising at least one sample cavity, the sample cavity is sized and shaped to hold a predetermined milk sample volume, and an overflow channel coupled to at least one outlet of the at least one sample cavity for diverting excess sample out of the sample cavity and into the overflow channel;
wherein the monitoring compartment couples with the at least one sample holding chamber so that the open base of the shell surrounds the sample holding chamber to create a milk sample enclosure such that at least part of the sensing portion of the sensing module is positioned within the sample cavity, and wherein the system is configured to monitor milk at all stages of breastfeeding and provide at least one status value relating to the breastfeeding efficiency in the human subject.

2. The system of claim 1, wherein the milk sample enclosure is configured to hold between about 200 microliters and up to about 500 microliters of milk, wherein the enclosure is defined by the walls of the milk sample cavity and a top roof of the shell base, and wherein
the enclosure, when formed, accommodates a volume of milk in which the entire sensing portion of the sensing module is immersed within the sample cavity, and wherein the at least one sample holding chamber comprises a base, the base comprising a walled ridge to receive the perimeter of the open base of the shell of the monitoring compartment and couples the monitoring compartment to the at least one sample holding compartment.

3. The system of claim 1, wherein the conductivity measurement of the milk sample is adjusted according to the milk measured temperature, affording a conductivity output that corresponds to ambient temperature.

4. The system of claim 1, wherein there are two electrodes, the two electrodes are disposed adjacent to each other, and the temperature sensor is disposed between the two electrodes to thereby form a triangle like structure allowing a compact configuration for optimal coupling and fit within the milk sample cavity.

5. The system of claim 1, wherein the volume sufficiency sensor comprises a conductive casing covering the temperature sensor, the casing is electrically wired to one of the conductivity electrodes, wherein the conductivity electrodes are immersed deeper into the milk sample than the temperature sensor and casing, and wherein when the conductive casing is in contact with the milk sample, the circuit is closed between the conductive casing and the at least one conductivity electrode facilitating a sufficient sample volume signal and when the conductive casing does not contact a volume of milk sample, the circuit between the conductive casing and the conductivity electrode is not closed facilitating an insufficient sample volume signal.

6. The system of claim 1, wherein the processing unit is configured to execute computer readable instructions to cause the monitoring device to measure the milk related parameters, analyze the measured parameters, trigger a visual and/or an audio output relating to the measurements, and provide insights for improving breastfeeding efficiency, within up to a few minutes.

7. The system of claim 1, wherein the monitoring compartment comprises a communication module configured to transmit the at least one milk related parameter to an external computing device configured to receive the measurements from the monitoring compartment, to display, store, and analyze the data, wherein the computing device comprising a designated program for monitoring and displaying milk related parameters and data communicated from the monitoring device.

8. The system of claim 1, configured to provide for one of the breasts, for each breast, or for both breasts in combination an output value relating to the breastfeeding efficiency status, the output value is selected from: a milk conductivity measurement, a milk maturation score reflecting milk sample maturation percent within the full range from initial colostrum to fully mature milk, a milk estimated sodium, a predicted milk sample age after birth, and a breastfeeding progress rate.

9. The system of claim 8, configured to compare at least one of the breastfeeding efficiency status values to a corresponding reference dataset for each day or day range after birth, and trigger an alarm or a visual output when the at least one breastfeeding efficiency status value is outside of a range, exceeds, or is below a predetermined threshold of the corresponding reference dataset.

10. The system of claim 9, wherein values that fail to meet a predetermined threshold are indicative of a delayed lactation and wherein values that correspond to another predetermined threshold or are improved parameters are indicative of an advanced lactation state.

11. The system of claim 10, wherein delayed lactation comprises a condition selected from low milk supply, delayed lactogenesis, secondary lactation insufficiency, low milk supply, lactation failure, primary lactation failure, insufficient glandular tissue.

12. The system of claim 8, configured to compare the at least one of the breastfeeding efficiency status values between one breast and the other breast or between two breastfeeding efficiency status values in previously stored data from the same breast of a lactating subject, over a period of days, or between one breast and a reference value further configured to provide an indication of breast health issue wherein the breast health issue is determined when the difference in measurements between the breastfeeding efficiency status values exceeds a predetermined threshold and when pain is recorded in the system for the breast that exhibited lower breastfeeding efficiency status values relative to the compared status value, and wherein the system triggers an alarm or a visual output when there is an indication regarding a breast health issue in the subject wherein the breast health issue is selected from a group consisting of breast inflammation, restricted milk flow, engorgement, ductal infection, mastitis, breast infection, and breast candida.

13. The system of claim 1, further configured to calculate an estimated breastmilk protein content in the milk sample utilizing the measured conductivity and a correlation between protein content and conductivity measurements in a pre-defined empirical reference early postpartum dataset.

14. The system of claim 1, further configured to trigger at least one of an alarm or a visual output indicating a technical error selected from the group consisting of: low battery, a milk sample volume insufficiency, a poor communication signal, a measurement that is outside of a range or exceeds a predetermined threshold values of a corresponding reference dataset, and a difference between a measurement and at least one previous measurements from the same subject that exceeds a predetermined threshold values.

15. A method for monitoring breast milk sample of a human subject at various stages of breastfeeding from colostrum through transition to fully mature milk, the method comprising:
obtaining a small volume of sample of breast milk from a lactating human subject;
placing the sample in a cavity of a milk sample holding chamber;
measuring in the milk sample a plurality of milk related parameters comprising conductivity and/or resistivity, temperature and sufficiency of volume in the cavity of the milk sample holding chamber;
receiving a conductivity and/or a resistivity measurement from a sensor arranged to measure milk related parameters in said milk sample;
receiving a milk sample quantity sufficiency signal from a sensor arranged to measure the milk sample level sufficiency within a milk sample holding chamber during a measurement;
receiving a milk sample temperature measurement from a sensor arranged to measure the milk sample temperature during a measurement; and
identifying at least one breastfeeding efficiency status values based upon a sufficient milk sample level and the at least one measurement relating to the conductivity and/or resistivity of the milk sample, wherein the conductivity and/or resistivity measurement is adjusted to afford a conductivity output that corresponds to a predefined temperature, by analyzing data related to the milk related parameters and providing an output of the breastfeeding status value.

16. The method of claim 15, wherein the breastfeeding efficiency status is selected from a group consisting of: a milk conductivity measurement, a milk maturation score reflecting milk sample maturation percent within the full range from initial colostrum to fully mature milk, a milk estimated sodium, a predicted milk sample age after birth, and a breastfeeding progress rate.

17. The method of claim 16, further comprising comparing the at least one of the breastfeeding status values to a reference dataset corresponding for each day or day range after birth and alerting when the at least one breastfeeding efficiency status value is outside of a range, exceeds, or is below a predetermined threshold of the corresponding reference dataset.

18. The method of claim 17, further comprising indicating a delayed lactation when said values are out of the predetermined threshold, and indicating an advanced lactation state when the values correspond to the threshold or are improved values comparing to the threshold, optionally wherein delayed lactation comprises a condition selected from low milk supply, delayed lactogenesis, secondary lactation insufficiency, low milk supply, lactation failure, primary lactation failure, insufficient glandular tissue.

19. The method of claim 16, further comprising calculating a breastfeeding progress rate by computing the difference and/or ratio between the at least one of the breastfeeding efficiency status values to a corresponding previously stored data from the same lactating subject, over a period of days to calculate a progress rate, and triggering an alarm or a visual output when the difference and/or ratio between the least one breastfeeding efficiency status values is outside of a range or exceeds, or fails to meet a predetermined threshold relative to the corresponding reference dataset for the specific day or day range after birth.

20. The method of claim 16, further comprising comparing the at least one of the breastfeeding status values between one breast and the other breast or between different days in the same breast or between one breast and a predetermined reference value, and triggering an alert or a visual output when the difference in measurements between the values exceeds a predetermined threshold.

21. The method of claim 16, further comprising calculating a predicted milk sample age after birth by matching the calculated milk maturation score or the measured milk conductivity to a corresponding reference dataset calculated for each day or range of days after birth, presenting the predicted milk sample age to the subject and/or triggering an alarm or a visual output when the predicted milk sample age value does not match the actual day after birth, indicating a delay in breastfeeding progress.

22. The method of claim 20, further comprising providing an indication of a breast health issue wherein the breast health issue is determined when the difference in measurements between the two measurements exceeds a predetermined threshold and the subject experiences pain in the breast that exhibits lower breastfeeding efficiency status values relative to the compared value, and wherein the system triggers an alarm or a visual output when there is an indication regarding a breast health issue in the subject, wherein the breast health issue is selected from a group consisting of breast inflammation, restricted milk flow, engorgement, ductal infection, mastitis, breast infection, breast candida, and a combination thereof.

23. The method of claim 15, further comprises calculating an estimated breastmilk protein content in the milk sample utilizing the measured conductivity and a correlation between protein content and conductivity measurements in a pre-defined empirical reference early postpartum dataset.

24. The method of claim 18, comprising providing insights for improving breastfeeding efficiency according to the measured breastfeeding efficiency status value.

25. A computer system configured for measuring indicators in human milk sample in different volumes from initial colostrum to fully mature milk, for use of monitoring breastfeeding efficiency, the system comprising one or more processing units, and one or more computer readable media storing executable instructions, and at least one sensor-set composed of sensor arranged to measure a conductivity and/or a resistivity, sensor indicative of sufficiency of human milk sample volume and sensor arranged to measure the human milk sample temperature, wherein execution thereof by the at least one processing unit configures the computer system to:
a. process and receive any of a conductivity and/or a resistivity measurement from the at least one sensor arranged to measure a conductivity and/or a resistivity in a human milk sample taken from at least one breast of a lactating person, wherein the conductivity and/or a resistivity measurement is performed in a milk sample holding chamber;
b. process and receive a milk sample volume signal from the at least one sensor indicative of sufficiency of human milk sample volume in the milk sample holding chamber during a measurement;
c. process and receive a milk sample temperature measurement from the at least one sensor arranged to measure the human milk sample temperature in the milk sample holding chamber during a measurement, and
d. determine an adjusted conductivity and/or adjusted resistivity according to the temperature measurement and milk sample volume signal measured by the sensor set in the milk sample holding chamber during a measurement to provide an adjusted conductivity and/or adjusted resistivity measurement, and
e. generate an output signal representing a breastfeeding efficiency status value, wherein the a breastfeeding efficiency status value includes the milk adjusted conductivity and/or adjusted resistivity measurement or computations thereof generating one or more additional status value selected from a group consisting of: a milk maturation percent reflecting milk sample maturation within the full range from initial colostrum to fully mature milk, a predicted milk sample age after birth, a breastfeeding progress rate, and a combination thereof;
f. compare said adjusted conductivity and/or adjusted resistivity or status value to a reference dataset comprising reference values of a population of lactating females corresponding to each day or day range postpartum; and
g. trigger an alarm or a visual output when the adjusted conductivity and/or adjusted resistivity or breastfeeding efficiency status value is outside of a range, exceeds, or is below a threshold of said dataset of reference values;
